(19) United States
(12) Patent Application Publication
(10) Pub. No.: US 2025/0115763 A1
(43) Pub. Date: Apr. 10, 2025

(54) COMPOUND, COMPOSITION, FILM, METHOD FOR PRODUCING COLORING COMPOSITION, AND METHOD FOR MANUFACTURING LAMINATE FOR DISPLAY ELEMENT

(71) Applicant: FUJIFILM Corporation, Tokyo (JP)

(72) Inventors: Masatoshi MIZUMURA, Shizuoka (JP); Daisuke ARIOKA, Shizuoka (JP); Masaya SUZUKI, Shizuoka (JP)

(73) Assignee: FUJIFILM Corporation, Tokyo (JP)

(21) Appl. No.: 18/985,034

(22) Filed: Dec. 18, 2024

Related U.S. Application Data
(63) Continuation of application No. PCT/JP2023/023015, filed on Jun. 21, 2023.

(30) Foreign Application Priority Data
Jun. 30, 2022 (JP) 2022-106689
Feb. 3, 2023 (JP) 2023-015690

(51) Int. Cl. C09B 7/08; C07D 405/04; C08K 5/3417; C09D 4/00; C09D 7/63; G02F 1/1335

(52) U.S. Cl. CPC C09B 7/08; C07D 405/04; C09D 4/00; C09D 7/63; C08K 5/3417; G02F 1/133512

(57) ABSTRACT

Provided are a compound represented by Formula (1), a composition and a film containing the compound, and a method for producing a coloring composition and a method for manufacturing a laminate for a display element using the compound. [in Formula (1), X¹, X², X³, X⁴, Y¹, and Y² each independently represent an oxygen atom, a sulfur atom, or N-L¹, L¹ represents a hydrogen atom, an alkyl group, an acyl group, an alkoxycarbonyl group, or an aminocarbonyl group, R¹, R², R³, and R⁴ each independently represent a hydrogen atom, —O-L², —OCO-L³, —S-L², or —OSO-L³, L² represents a hydrogen atom or an alkyl group and L³ represents an alkyl group or an amino group, provided that at least one of R¹ or R² represents a hydrogen atom and at least one of R³ or R⁴ represents a hydrogen atom, and A, B, and C each independently represent an aromatic ring]

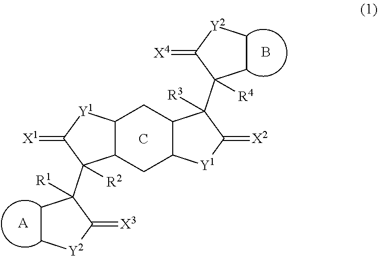

### CROSS-REFERENCE TO RELATED APPLICATIONS

This application is a continuation application of International Application No. PCT/JP2023/023015, filed Jun. 21, 2023, which is incorporated herein by reference. Further, this application claims priority from Japanese Patent Application No. 2022-106689, filed Jun. 30, 2022, and Japanese Patent Application No. 2023-015690, filed Feb. 3, 2023, which are incorporated herein by reference.

### BACKGROUND OF THE INVENTION

### 1. Field of the Invention

The present disclosure relates to a compound, a composition, a film, a method for producing a coloring composition, and a method for manufacturing a laminate for a display element.

### 2. Description of the Related Art

In the related art, a black pigment such as carbon black has been widely used in a light shielding film which shields light in a wide wavelength range. In general, the black pigment is a coloring material which absorbs light in a wide wavelength range and has excellent light shielding properties, but also absorbs light in an ultraviolet (UV) range. Therefore, for example, in a case where a film containing the black pigment is irradiated with UV light to form a pattern, the incident UV light may be gradually attenuated in a film thickness direction, making it difficult to form the pattern. Such a phenomenon tends to occur more easily as the thickness of the film increases.

Here, JP2019-179111A discloses a composition for forming a partition wall, which contains the black pigment. In addition, WO2015/016294A discloses a coloring curable resin composition containing a bisbenzofuranone compound having a specific structure as a black coloring material of a black matrix.

### SUMMARY OF THE INVENTION

JP2019-179111A discloses a liquid crystal display device including a high partition wall, and the black pigment is used as a coloring material in a film forming the partition wall. In JP2019-179111A, since a film having a thickness is formed by allowing UV light to be transmitted to a lower part of a coating film of a composition for forming a partition wall by suppressing a concentration of the black pigment, it is difficult to ensure sufficient light shielding properties in a case where an attempt is made to form a thicker film, and thus there is room for improvement.

There is a demand for the development of a new technique as a substitute for the technique of using the black pigment in the related art.

An object to be achieved by one embodiment of the present disclosure is to provide a novel compound capable of achieving both transmission and shielding of light.

An object to be achieved by another embodiment of the present disclosure is to provide a composition and a film, which contain the above-described compound.

An object to be achieved by still another embodiment of the present disclosure is to provide a method for producing a coloring composition and a method for manufacturing a laminate for a display element, each using the above-described compound.

As a result of extensive studies, the present inventors have found a novel compound that is colored due to a change in structure caused by heat, and have completed the invention according to the present disclosure.

Specific methods for achieving the above-described objects include the following aspects.

<1> A compound represented by Formula (1).

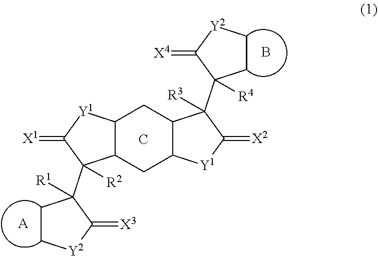

In Formula (1), X¹, X², X³, X⁴, Y¹, and Y² each independently represent an oxygen atom, a sulfur atom, or N-L¹, L¹ represents a hydrogen atom, an alkyl group, an acyl group, an alkoxycarbonyl group, or an aminocarbonyl group, R¹, R², R³, and R⁴ each independently represent a hydrogen atom, —O-L², —OCO-L³, —S-L², or —OSO-L³, L² represents a hydrogen atom or an alkyl group and L³ represents an alkyl group or an amino group, provided that at least one of R¹ or R² represents a hydrogen atom and at least one of R³ or R⁴ represents a hydrogen atom, and A, B, and C each independently represent an aromatic ring.

<2> The compound according to <1>,
in which, in Formula (1), X¹, X², X³, and X⁴ are oxygen atoms, one of R¹ or R² is a hydrogen atom and the other is a hydroxy group, and one of R³ or R⁴ is a hydrogen atom and the other is a hydroxy group.

<3> The compound according to <1>,
in which, in Formula (1), X¹, X², X³, and X⁴ are oxygen atoms, and R¹, R², R³, and R⁴ are hydrogen atoms.

<4> The compound according to any one of <1> to <3>,
in which the compound is a coloring agent precursor.

<5> A composition comprising:
a compound represented by Formula (1).

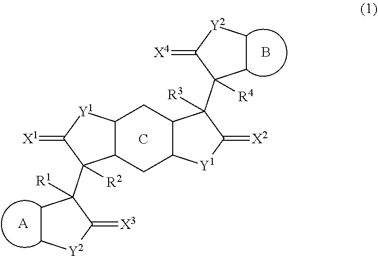

In Formula (1), X¹, X², X³, X⁴, Y¹, and Y² each independently represent an oxygen atom, a sulfur atom, or N-L¹, L¹ represents a hydrogen atom, an alkyl group, an acyl group, an alkoxycarbonyl group, or an aminocarbonyl group, R¹, R², R³, and R⁴ each independently represent a hydrogen atom, —O-L², —OCO-L³, —S-L², or —OSO-L³, L² represents a hydrogen atom or an alkyl group and L³ represents an alkyl group or an amino group, provided that at least one of R¹ or R² represents a hydrogen atom and at least one of R³ or R⁴ represents a hydrogen atom, and A, B, and C each independently represent an aromatic ring.

<6> The composition according to <5>, further comprising:
a resin.

<7> A film comprising:
a compound represented by Formula (1).

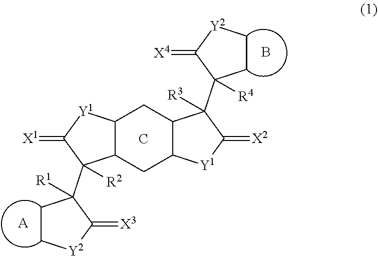

In Formula (1), X¹, X², X³, X⁴, Y¹, and Y² each independently represent an oxygen atom, a sulfur atom, or N-L¹, L¹ represents a hydrogen atom, an alkyl group, an acyl group, an alkoxycarbonyl group, or an aminocarbonyl group, R¹, R², R³, and R⁴ each independently represent a hydrogen atom, —O-L², —OCO-L³, —S-L², or —OSO-L³, L² represents a hydrogen atom or an alkyl group and L³ represents an alkyl group or an amino group, provided that at least one of R¹ or R² represents a hydrogen atom and at least one of R³ or R⁴ represents a hydrogen atom, and A, B, and C each independently represent an aromatic ring.

<8> A method for producing a coloring composition, comprising:
a step A of obtaining a composition containing a compound represented by Formula (1) and a resin; and
a step B of heating the composition.

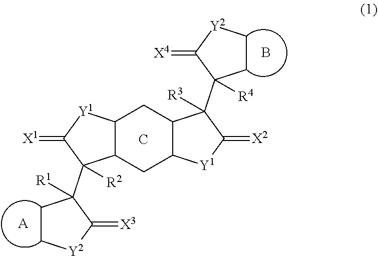

In Formula (1), X¹, X², X³, X⁴, Y¹, and Y² each independently represent an oxygen atom, a sulfur atom, or N-L¹, L¹ represents a hydrogen atom, an alkyl group, an acyl group, an alkoxycarbonyl group, or an aminocarbonyl group, R¹, R², R³, and R⁴ each independently represent a hydrogen atom, —O-L², —OCO-L³, —S-L², or —OSO-L³, L² represents a hydrogen atom or an alkyl group and L³ represents an alkyl group or an amino group, provided that at least one of R¹ or R² represents a hydrogen atom and at least one of R³ or R⁴ represents a hydrogen atom, and A, B, and C each independently represent an aromatic ring.

<9> The method for producing a coloring composition according to <8>,
in which the composition further contains a polymerization initiator.

<10> The method for producing a coloring composition according to <8> or <9>,
in which the composition further contains a polymerizable monomer.

<11> The method for producing a coloring composition according to <8>,
in which the composition further contains an acid generator, and the resin includes an acid-decomposable resin.

<12> A method for manufacturing a laminate for a display element, comprising:
a step of producing a coloring composition by the method for producing a coloring composition according to any one of <8> to <11>.

According to one embodiment of the present disclosure, there is provided a novel compound capable of achieving both transmission and shielding of light.

According to another embodiment of the present disclosure, there are provided a composition and a film, which contain the above-described compound.

According to still another embodiment of the present disclosure, there are provided a method for producing a coloring composition and a method for manufacturing a laminate for a display element, each using the above-described compound.

### BRIEF DESCRIPTION OF THE DRAWINGS

FIG. 1A is a graph showing an absorption spectrum of a THF solution of a compound (100).

FIG. 1B is a graph showing an absorption spectrum of a THF solution of a compound (1).

FIG. 1C is a graph showing an absorption spectrum of a THF solution of a compound (1H).

### DESCRIPTION OF THE PREFERRED EMBODIMENTS

Hereinafter, the present disclosure will be described in detail. The description of the requirements set forth below may be based on representative embodiments of the present disclosure, but the present disclosure is not limited to such embodiments. Within the scope of the purpose of the present disclosure, modifications can be made as appropriate.

In the present disclosure, a numerical range expressed using “to” means a range including numerical values before and after “to” as a lower limit value and an upper limit value. In a numerical range described in a stepwise manner in the present disclosure, an upper limit or a lower limit described in a certain numerical range may be replaced with an upper limit or a lower limit in another numerical range described in a stepwise manner. In addition, in a numerical range described in the present disclosure, an upper limit or a lower limit described in a certain numerical range may be replaced with a value described in Examples.

In the present disclosure, upon referring to an amount of each component in a composition, the amount means a total amount of a plurality of components present in the composition unless otherwise specified, in a case where a plurality of substances corresponding to individual components are present in the composition.

In the present disclosure, a combination of two or more preferred aspects is a more preferred aspect.

In the present disclosure, “solid content” means a component excluding a solvent, and “solvent” means water and an organic solvent.

In the present disclosure, the meaning of “step” includes not only an independent step but also a step whose intended purpose is achieved even in a case where the step is not clearly distinguished from other steps.

In the present disclosure, “(meth)acryl” is a term including both “acryl” and “methacryl”; “(meth)acrylate” is a term including both “acrylate” and “methacrylate”; “(meth)acryloyl” is a term including both “acryloyl” and “methacryloyl”; and “(meth)allyl” is a term including both “methallyl” and “allyl”.

In the present disclosure, “n-” means normal, “s-” means secondary, and “t-” means tertiary.

In the present disclosure, “light” refers to, for example, ultraviolet light, visible light, infrared light, or the like.

In the present disclosure, the “ultraviolet light” refers to light in a wavelength range of 200 nm or more and less than 400 nm, the “visible light” refers to light in a wavelength range of 400 nm or more and less than 780 nm, and the “infrared light” refers to light of 780 nm or more and less than 1,000 nm.

In the present disclosure, in a case where there is a molecular weight distribution, a molecular weight represents a weight-average molecular weight (Mw; the same applies hereinafter), unless otherwise noted.

The weight-average molecular weight (Mw) in the present disclosure is measured by gel permeation chromatography (GPC).

The measurement by GPC is performed using HLC (registered trademark)-8220GPC [manufactured by Tosoh Corporation] as a measuring device, three columns of TSKgel (registered trademark) Super HZ2000 [4.6 mm ID×15 cm, manufactured by Tosoh Corporation], TSKgel (registered trademark) Super HZ4000 [4.6 mm ID×15 cm, manufactured by Tosoh Corporation] and TSKgel (registered trademark) Super HZ—H [4.6 mm ID×15 cm, manufactured by Tosoh Corporation] which are connected in series, and N-methylpyrrolidone (NMP) as an eluent. A differential refractive index (RI) detector as a detector is used for performing the GPC under the measurement conditions: a sample concentration of 0.3% by mass, a flow rate of 0.35 mL/min, a sample injection volume of 10 μL, and a measurement temperature of 40° C. The calibration curve is prepared using 6 samples of “F-80”, “F-20”, “F-4”, “F-2”, “A-5000”, and “A-1000”, which are “Standard Sample TSK standard, polystyrene” manufactured by Tosoh Corporation.

Regarding a term of a group (atomic group) in the present disclosure, a term with no description of “substituted” and “unsubstituted” includes both a group not having a substituent and a group having a substituent. For example, the concept of “alkyl group” includes not only an alkyl group not having a substituent (unsubstituted alkyl group) but also an alkyl group having a substituent (substituted alkyl group).

The “substituent” in the present disclosure is not particularly limited, and for example, it can be arbitrarily selected from a substituent group consisting of a halogen group, a hydroxy group, an alkyl group, a cycloalkyl group, an alkenyl group, a cycloalkenyl group, an alkynyl group, an aryl group, a heterocyclic group, an alkoxy group, an aryloxy group, a heterocyclicoxy group, a sulfo group, an acyl group, an alkoxycarbonyl group, an aryloxycarbonyl group, a carboxy group, a carbamoyl group, an acyloxy group, a carbamoyloxy group, an alkoxycarbonyloxy group, aryloxycarbonyloxy, a cyano group, a nitro group, an amino group (including an anilino group), an acylamino group, an aminocarbonylamino group, an alkoxycarbonylamino group, an aryloxycarbonylamino group, a sulfamoylamino group, an alkylsulfonylamino group, an arylsulfonylamino group, a mercapto group, an alkylthio group, an arylthio group, a heterocyclicthio group, a sulfamoyl group, an alkylsulfinyl group, an arylsulfinyl group, an alkylsulfonyl group, an arylsulfonyl group, an arylazo group, a heterocyclic azo group, an imide group, a phosphino group, a phosphinyl group, a phosphinyloxy group, and a phosphinylamino group.

More specific examples of the substituent in the present disclosure include: a halogen group (for example, a fluorine group, a chlorine group, a bromine group, and an iodine group);
an alkyl group (a linear, branched, or cyclic alkyl group having 1 to 10, preferably 1 to 6 carbon atoms; for example, a methyl group, an ethyl group, an n-propyl group, an isopropyl group, a t-butyl group, an n-octyl group, a 2-chloroethyl group, a 2-cyanoethyl group, and a 2-ethylhexyl group);
a cycloalkyl group (preferably, a cyclopropyl group or a cyclopentyl group); an alkenyl group (a linear, branched, or cyclic alkenyl group having 2 to 10, preferably 2 to 6 carbon atoms; for example, a vinyl group, an allyl group, and a prenyl group); a cycloalkenyl group (preferably a cyclopenten-1-yl group);
an alkynyl group (an alkynyl group having 2 to 10, preferably 2 to 6 carbon atoms; for example, an ethynyl group and a propargyl group);
an aryl group (an aryl group having 6 to 12, preferably 6 to 8 carbon atoms; for example, a phenyl group, a p-tolyl group, a naphthyl group, a 3-chlorophenyl group, and a 2-aminophenyl group);
a heterocyclic group (a monovalent group having 1 to 12, preferably 2 to 6 carbon atoms, which is obtained by removing one hydrogen atom from a 5-membered or 6-membered aromatic or non-aromatic heterocyclic compound; for example, a 1-pyrazolyl group, a 1-imidazolyl group, a 2-furyl group, a 2-thienyl group, a 4-pyrimidinyl group, and a 2-benzothiazolyl group);
a cyano group;
a hydroxy group;
a nitro group;
an alkoxy group (a linear, branched, or cyclic alkoxy group having 1 to 10, preferably 1 to 6 carbon atoms; for example, a methoxy group, an ethoxy group, an isopropoxy group, a t-butoxy group, a cyclopentyloxy group, a 2-buten-1-yloxy group, and a 2-methoxyethoxy group);
an aryloxy group (an aryloxy group having 6 to 12, preferably 6 to 8 carbon atoms; for example, a phenoxy group, a 2-methylphenoxy group, a 4-t-butylphenoxy group, and a 3-nitrophenoxy group);
a heterocyclic oxy group (a heterocyclic oxy group having 1 to 12, preferably 2 to 6 carbon atoms; for example, a 1-phenyltetrazol-5-oxy-2-tetrahydropyranyloxy group);
an acyloxy group (an acyloxy group having 1 to 12, preferably 1 to 8 carbon atoms; for example, a formyloxy group, an acetyloxy group, a pivaloyloxy group, a benzoyloxy group, and a p-methoxycarbonyloxy group);
a carbamoyloxy group (a carbamoyloxy group having 1 to 10, preferably 1 to 6 carbon atoms; for example, an N,N-dimethylcarbamoyloxy group, an N,N-diethylcarbamoyloxy group, a morpholinocarbonyloxy group, and an N,N-octylcarbamoyloxy group);
an alkoxycarbonyloxy group (an alkoxycarbonyloxy group having 2 to 10, preferably 2 to 6 carbon atoms; for example, a methoxycarbonyloxy group, an ethoxycarbonyloxy group, a t-butoxycarbonyloxy group, and an n-octyloxycarbonyloxy group);
an aryloxycarbonyloxy group (an aryloxycarbonyloxy group having 7 to 12, preferably 7 to 10 carbon atoms; for example, a phenoxycarbonyloxy group and a p-methoxyphenoxycarbonyloxy group);
an amino group (including an amino group, an alkylamino group having 1 to 10, preferably 1 to 6 carbon atoms, an anilino group having 6 to 12, preferably 6 to 8 carbon atoms, and a heterocyclic amino group having 1 to 12, preferably 2 to 6 carbon atoms; for example, an amino group, a methylamino group, a dimethylamino group, an anilino group, an N-methyl-anilino group, a diphenylamino group, an imidazol-2-ylamino group, and a pyrazol-3-ylamino group);
an acylamino group (including an alkylcarbonylamino group having 1 to 10, preferably 1 to 6 carbon atoms, an arylcarbonylamino group having 6 to 12, preferably 6 to 8 carbon atoms, and a heterocyclic carbonylamino group having 2 to 12, preferably 2 to 6 carbon atoms; for example, a formylamino group, an acetylamino group, a pivaloylamino group, a benzoylamino group, a pyridine-4-carbonylamino group, and a thiophene-2-carbonylamino group);
an aminocarbonylamino group (an aminocarbonylamino group having 1 to 12, preferably 1 to 6 carbon atoms; for example, a carbamoylamino group, an N,N-dimethylaminocarbonylamino group, an N,N-diethylaminocarbonylamino group, and a morpholin-4-ylcarbonylamino group);
an alkoxycarbonylamino group (an alkoxycarbonylamino group having 2 to 10, preferably 2 to 6 carbon atoms; for example, a methoxycarbonylamino group, an ethoxycarbonylamino group, and a t-butoxycarbonylamino group);
an aryloxycarbonylamino group (an aryloxycarbonylamino group having 7 to 12, preferably 7 to 9 carbon atoms; for example, a phenoxycarbonylamino group, a p-chlorophenoxycarbonylamino group, and a 4-methoxyphenoxycarbonylamino group);
a sulfamoylamino group (a sulfamoylamino group having 0 to 10, preferably 0 to 6 carbon atoms; for example, a sulfamoylamino group, an N,N-dimethylaminosulfonylamino group, and an N-(2-hydroxyethyl)sulfamoylamino group);
an alkylsulfonylamino group (an alkylsulfonylamino group having 1 to 10, preferably 1 to 6 carbon atoms; for example, a methylsulfonylamino group and a butylsulfonylamino group);
an arylsulfonylamino group (an arylsulfonylamino group having 6 to 12, preferably 6 to 8 carbon atoms; for example, a phenylsulfonylamino group, a 2,3,5-trichlorophenylsulfonylamino group, and a p-methylphenylsulfonylamino group);
a mercapto group;
an alkylthio group (an alkylthio group having 1 to 10, preferably 1 to 6 carbon atoms; for example, a methylthio group, an ethylthio group, and a butylthio group);
an arylthio group (an arylthio group having 6 to 12, preferably 6 to 8 carbon atoms; for example, a phenylthio group, a p-chlorophenylthio group, and a m-methoxythio group);
a heterocyclic thio group (a heterocyclic thio group having 2 to 10, preferably 1 to 6 carbon atoms; for example, a 2-benzothiazolylthio group and a 1-phenyltetrazol-5-ylthio group);
a sulfamoyl group (a sulfamoyl group having 0 to 10, preferably 0 to 6 carbon atoms; for example, a sulfamoyl group, an N-ethylsulfamoyl group, an N,N-dimethylsulfamoyl group, an N-acetylsulfamoyl group, and an N-benzoylsulfamoyl group);
an alkylsulfinyl group (an alkylsulfinyl group having 1 to 10, preferably 1 to 6 carbon atoms; for example, a methylsulfinyl group and an ethylsulfinyl group);
an arylsulfinyl group (an arylsulfinyl group having 6 to 12, preferably 6 to 8 carbon atoms; for example, a phenylsulfinyl group and a p-methylphenylsulfinyl group);
an alkylsulfonyl group (an alkylsulfonyl group having 1 to 10, preferably 1 to 6 carbon atoms; for example, a methylsulfonyl group and an ethylsulfonyl group);
an arylsulfonyl group (arylsulfonyl group having 6 to 12, preferably 6 to 8 carbon atoms; for example, a phenylsulfonyl group and a p-chlorophenylsulfonyl group); a sulfo group;
an acyl group (a formyl group, an alkylcarbonyl group having 2 to 10, preferably 2 to 6 carbon atoms, or an arylcarbonyl group having 7 to 12, preferably 7 to 9 carbon atoms; for example, an acetyl group, a pivaloyl group, a 2-chloroacetyl group, a benzoyl group, and a 2,4-dichlorobenzoyl group);
an alkoxycarbonyl group (an alkoxycarbonyl group having 2 to 10, preferably 2 to 6 carbon atoms; for example, a methoxycarbonyl group, an ethoxycarbonyl group, a t-butoxycarbonyl group, and an isobutyloxycarbonyl group);
an aryloxycarbonyl group (an aryloxycarbonyl group having 7 to 12, preferably 7 to 9, carbon atoms; for example, a phenoxycarbonyl-2-chlorophenoxycarbonyl group, a 3-nitrophenoxycarbonyl group, and a 4-t-butylphenoxycarbonyl group);
a carbamoyl group (a carbamoyl group having 1 to 10, preferably 1 to 6 carbon atoms; for example, a carbamoyl group, an N-methylcarbamoyl group, an N,N-dimethylcarbamoyl group, an N-(2-hydroxyethyl)carbamoyl group, and an N-(methylsulfonyl)carbamoyl group);
an arylazo group (an arylazo group having 6 to 12, preferably 6 to 8 carbon atoms; for example, a phenylazo group and a p-chlorophenylazo group);
a heterocyclic azo group (a heterocyclic azo group having 1 to 10, preferably 1 to 6 carbon atoms; for example, a pyrazol-3-ylazo group, a thiazol-2-ylazo group, and a 5-methylthio-1,3,4-thiadiazol-2-ylazo group);
an imido group (an imido group having 2 to 10, preferably 4 to 8 carbon atoms; for example, a succinimido group and a phthalimido group);
a phosphino group (a phosphinyl group having 2 to 12, preferably 2 to 6 carbon atoms; for example, a dimethylphosphino group, a diphenylphosphino group, and a methylphenoxyphosphino group);
a phosphinyl group (a phosphinyl group having 2 to 12, preferably 2 to 6 carbon atoms; for example, a phosphinyl group and a diethoxyphosphinyl group);
a phosphinyloxy group (a phosphinyloxy group having 2 to 12, preferably 2 to 6 carbon atoms; for example, a diphenoxyphosphinyloxy group and a dibutoxyphosphinyloxy group); and
a phosphinylamino group (a phosphinylamino group having 2 to 12, preferably 2 to 6 carbon atoms; for example, a dimethoxyphosphinylamino group and a dimethylaminophosphinylamino group).

In a case where these groups are groups which can be further substituted, these groups can further include a substituent. In a case where two or more of these groups are substituted with a substituent, the substituents may be the same or different from each other.

### [Compound Represented by Formula (1)]

The compound according to the present disclosure is a compound represented by Formula (1).

In a case where tautomers and/or geometric isomers exist with regard to the compound represented by Formula (1), the existing tautomers and/or geometric isomers are included in the compound represented by Formula (1).

The “tautomer” refers to, for example, a compound that exists as two or more isomers which can be readily interconverted from one to the other. Examples of the tautomer include an isomer which is generated by moving a proton bonded to one atom in a molecule to another atom, and an isomer which is generated by moving a valence localized in a specific atom in a molecule to another atom.

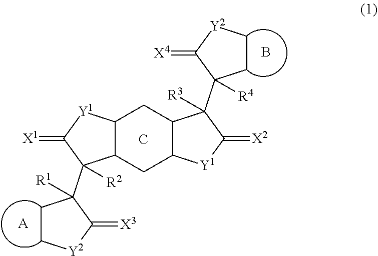

In Formula (1), X¹, X², X³, X⁴, Y¹, and Y² each independently represent an oxygen atom, a sulfur atom, or N-L¹, L¹ represents a hydrogen atom, an alkyl group, an acyl group, an alkoxycarbonyl group, or an aminocarbonyl group, R¹, R², R³, and R⁴ each independently represent a hydrogen atom, —O-L², —OCO-L³, —S-L², or —OSO-L³, L² represents a hydrogen atom or an alkyl group and L³ represents an alkyl group or an amino group, provided that at least one of R¹ or R² represents a hydrogen atom and at least one of R³ or R⁴ represents a hydrogen atom, and A, B, and C each independently represent an aromatic ring.

The compound according to the present disclosure is a novel compound capable of achieving both transmission and shielding of light. Since the compound according to the present disclosure is colorless, the compound can transmit light in a wide wavelength range. In addition, the compound according to the present disclosure has a characteristic of being colored by heating. The mechanism by which the compound according to the present disclosure is colored by heating is not clear, but the present inventors have considered as follows.

In a case of being heated, it is considered that the compound according to the present disclosure reacts with oxygen in the air and is oxidized to change the structure into an oxidant, and the oxidant is colored. The present inventors have considered that, for example, the compound according to the present disclosure reacts with oxygen in the air, R¹, R², R³, and R⁴ in Formula (1) are eliminated (for example, dehydration, de-alcoholization, and the like), a single bond between R¹ and R² and a single bond between R³ and R⁴ are changed to a double bond, and thus conjugation is extended, so that a wavelength of light to be absorbed changes to the visible range and the compound is colored.

For example, since black absorbs light in a wide wavelength range, in a case where a film containing a black coloring material is irradiated with UV light to form a pattern, the incident UV light is gradually attenuated in a film thickness direction, the UV light does not reach a deep portion of the film, and a pattern having a favorable shape is hardly obtained after development due to insufficient curing.

However, by using the compound according to the present disclosure, since the compound according to the present disclosure before heating is colorless, during exposure, the UV light is likely to travel from an incident surface of the film toward the bottom portion, and a photocuring reaction in the film thickness direction can be promoted. In addition, the film after exposure, containing the compound according to the present disclosure, can also be colored black by heating, for example. Therefore, with the compound according to the present disclosure, a pattern can be formed in a favorable shape and in a colored manner (preferably, colored black).

With the compound according to the present disclosure, it is possible to easily color an article by applying heat at a stage in which transmission of light is not necessary, without applying heat during a period in which transmission of light is necessary during exposure.

Details of the compound represented by Formula (1) will be described.

In Formula (1), X¹, X², X³, X⁴, Y¹, and Y² each independently represent an oxygen atom, a sulfur atom, or N-L¹.

X¹, X², X³, and X⁴ are preferably oxygen atoms.

In Formula (1), two Y¹'s may be the same or different from each other, but are preferably the same.

Y¹ is preferably an oxygen atom.

In Formula (1), two Y²'s may be the same or different from each other, but are preferably the same.

Y² is preferably N-L¹.

L¹ represents a hydrogen atom, an alkyl group, an acyl group, an alkoxycarbonyl group, or an aminocarbonyl group.

L¹ is preferably a hydrogen atom, an alkyl group, an acyl group, or an alkoxycarbonyl group; and more preferably an alkyl group, an acyl group, or an alkoxycarbonyl group.

The alkyl group represented by L¹ may have a substituent or may not have a substituent. The alkyl group represented by L¹ may be a linear alkyl group, a branched alkyl group, or an alkyl group having a cyclic structure.

The alkyl group represented by L¹ is preferably an alkyl group having 1 to 30 carbon atoms, and more preferably an alkyl group having 1 to 12 carbon atoms.

As the alkyl group represented by L¹, for example, an s-butyl group, an n-hexyl group, a 2-ethoxyethyl group, a methoxycarbonylmethyl group, an isopropyl group, an n-pentyl group, or a 2-ethylhexyl group is preferable.

The acyl group represented by L¹ is preferably an acyl group having 2 to 30 carbon atoms, and more preferably an acyl group having 2 to 15 carbon atoms.

As the acyl group represented by L¹, for example, an acetyl group, a 2-ethylhexanoyl group, a 3,3,5-trimethylhexanoyl group, a propionyl group, a butyryl group, an isobutyryl group, or a pivaloyl group is preferable.

The alkoxycarbonyl group represented by L¹ is preferably an alkoxycarbonyl group having 1 to 30 carbon atoms in an alkoxy moiety.

As the alkoxycarbonyl group represented by L¹, for example, a methoxycarbonyl group, an ethoxycarbonyl group, a butoxycarbonyl group, a t-butoxycarbonyl group, a 9-fluorenylmethyloxycarbonyl group, a benzyloxycarbonyl group, or a 2,2,2-trichloroethyloxycarbonyl group is preferable.

In Formula (1), R¹, R², R³, and R⁴ each independently represent a hydrogen atom, —O-L², —OCO-L³, —S-L², or —OSO-L³. However, at least one of R¹ or R² represents a hydrogen atom and at least one of R³ or R⁴ represents a hydrogen atom.

In a case where one of R¹ or R² is a hydrogen atom, the other is preferably a hydrogen atom or a hydroxy group (that is, —O-L² in which L² is a hydrogen atom), and more preferably a hydrogen atom.

In a case where one of R³ or R⁴ is a hydrogen atom, the other is preferably a hydrogen atom or a hydroxy group (that is, —O-L² in which L² is a hydrogen atom), and more preferably a hydrogen atom.

L² represents a hydrogen atom or an alkyl group.

L² is preferably a hydrogen atom.

The alkyl group represented by L² may have a substituent or may not have a substituent. The alkyl group represented by L² may be a linear alkyl group, a branched alkyl group, or an alkyl group having a cyclic structure.

The alkyl group represented by L² is preferably an alkyl group having 1 to 30 carbon atoms, and more preferably an alkyl group having 1 to 12 carbon atoms.

As the alkyl group represented by L², for example, a methyl group, an ethyl group, an n-propyl group, or a 2-ethylhexyl group is preferable.

L³ represents an alkyl group or an amino group.

The alkyl group represented by L³ may have a substituent or may not have a substituent. The alkyl group represented by L³ may be a linear alkyl group, a branched alkyl group, or an alkyl group having a cyclic structure.

The alkyl group represented by L³ is preferably an alkyl group having 1 to 30 carbon atoms, and more preferably an alkyl group having 1 to 12 carbon atoms.

As the alkyl group represented by L³, for example, a methyl group, an ethyl group, an n-propyl group, or a 2-ethylhexyl group is preferable.

A, B, and C each independently represent an aromatic ring.

The aromatic ring represented by A and the aromatic ring represented by B may be the same or different from each other. The aromatic ring represented by A and B may have a substituent or may not have a substituent. The aromatic ring represented by A and B may be, for example, an aromatic hydrocarbon ring, an aromatic heterocyclic ring, or a fused ring thereof.

In a case where the aromatic ring represented by A and B is an aromatic hydrocarbon ring, the aromatic hydrocarbon ring represented by A and B is preferably a 5-membered ring or a 6-membered ring, and more preferably a 6-membered ring.

In a case where the aromatic ring represented by A and B is an aromatic hydrocarbon ring, the aromatic hydrocarbon ring represented by A and B is preferably an aromatic hydrocarbon ring having 6 to 30 carbon atoms, more preferably an aromatic hydrocarbon ring having 6 to 20 carbon atoms, and still more preferably an aromatic hydrocarbon ring having 6 to 10 carbon atoms.

In a case where the aromatic ring represented by A and B is an aromatic hydrocarbon ring, the aromatic hydrocarbon ring represented by A is, for example, preferably a benzene ring, a naphthalene ring, or an anthracene ring, and more preferably a benzene ring.

In a case where the aromatic ring represented by A and B is an aromatic heterocyclic ring, the aromatic heterocyclic ring represented by A and B is preferably a 5-membered ring or a 6-membered ring, and more preferably a 5-membered ring.

In a case where the aromatic ring represented by A and B is an aromatic heterocyclic ring, the aromatic heterocyclic ring represented by A and B is preferably an aromatic heterocyclic ring including one or more heteroatoms selected from the group consisting of an oxygen atom, a sulfur atom, and a nitrogen atom in a ring. The number of heteroatoms in the aromatic heterocyclic ring is preferably 1 or 2, and more preferably 1.

In a case where the aromatic ring represented by A and B is an aromatic heterocyclic ring, the aromatic heterocyclic ring represented by A and B is, for example, preferably a thiophene ring, a furan ring, a pyrrole ring, an imidazole ring, a triazole ring, or a pyridine ring, and more preferably a thiophene ring.

The aromatic ring represented by C may have a substituent or may not have a substituent.

Examples of the aromatic ring represented by C include a benzene ring and a heterocyclic ring.

Examples of the heterocyclic ring include a pyridine ring and a pyrazine ring.

As the aromatic ring represented by C, a benzene ring is preferable.

In Formula (1), X¹, X², X³, X⁴, R¹, R², R³, and R⁴ are, for example, preferably the following aspect A, and more preferably the following aspect B.

Aspect A: aspect that X¹, X², X³, and X⁴ are oxygen atoms, at least one of R¹ or R² is a hydrogen atom and the other is a hydroxy group, and at least one of R³ or R⁴ is a hydrogen atom and the other is a hydroxy group

Aspect B: aspect that X¹, X², X³, and X⁴ are oxygen atoms, and R¹, R², R³, and R⁴ are hydrogen atoms

A preferred aspect of the compound represented by Formula (1) is an aspect that X¹, X², X³, and X⁴ are oxygen atoms, Y¹ and Y² are each independently an oxygen atom, a sulfur atom, or N-L¹, L¹ is a hydrogen atom, an alkyl group, an acyl group, or an alkoxycarbonyl group, one of R¹ or R² is a hydrogen atom and the other is a hydroxy group, one of R³ or R⁴ is a hydrogen atom and the other is a hydroxy group, A and B are each independently a benzene ring or a thiophene ring, and C is a benzene ring.

A more preferred aspect of the compound represented by Formula (1) is an aspect that X¹, X², X³, and X⁴ are oxygen atoms, Y¹ is an oxygen atom, Y² is N-L¹, L¹ is an alkyl group, an acyl group, or an alkoxycarbonyl group, R¹, R², R³, and R⁴ are hydrogen atoms, A and B are benzene rings, and C is a benzene ring.

Specific examples of the compound represented by Formula (1) are described below, but the present disclosure is not limited to these examples. “Me” represents methyl.

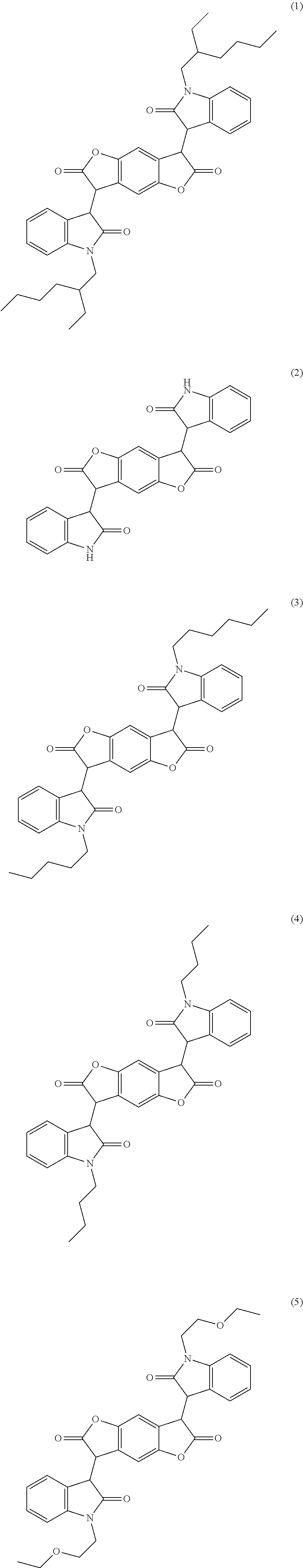

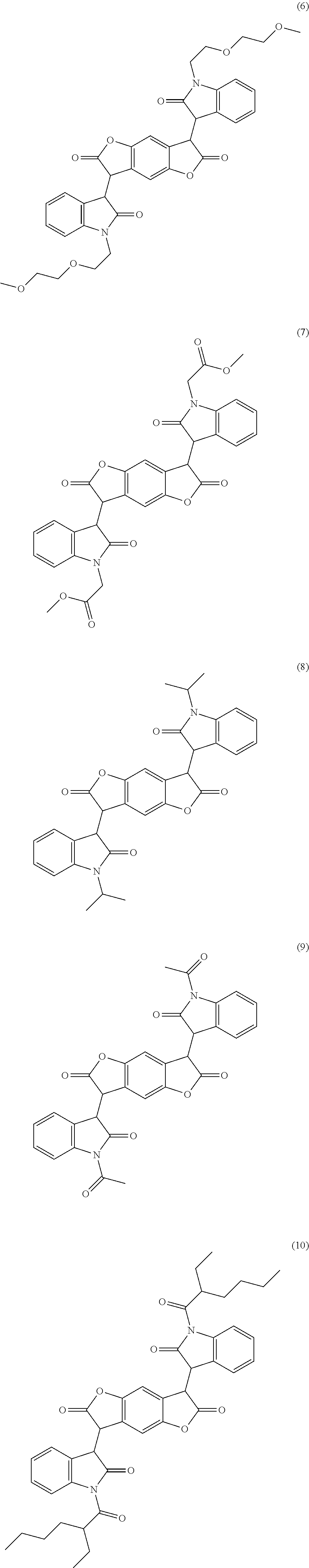

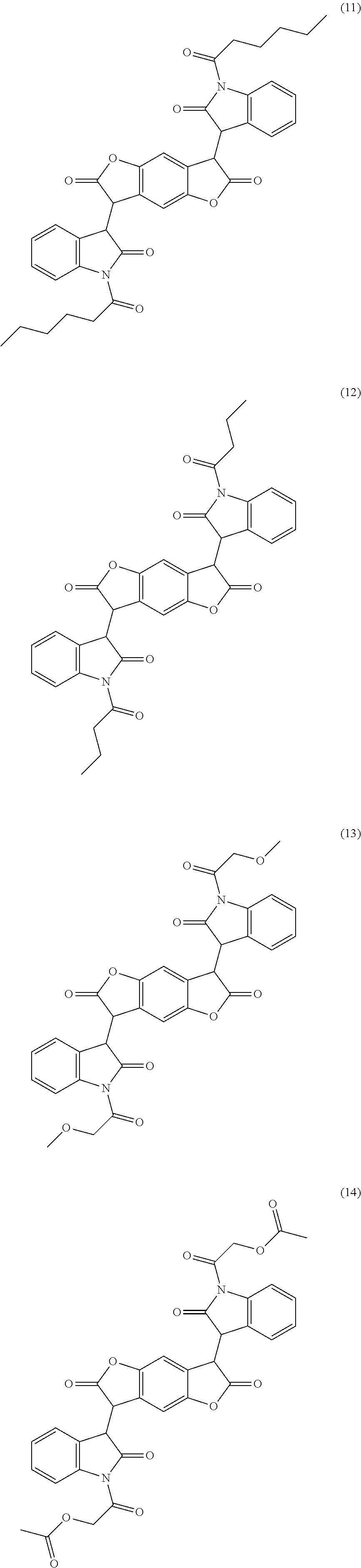

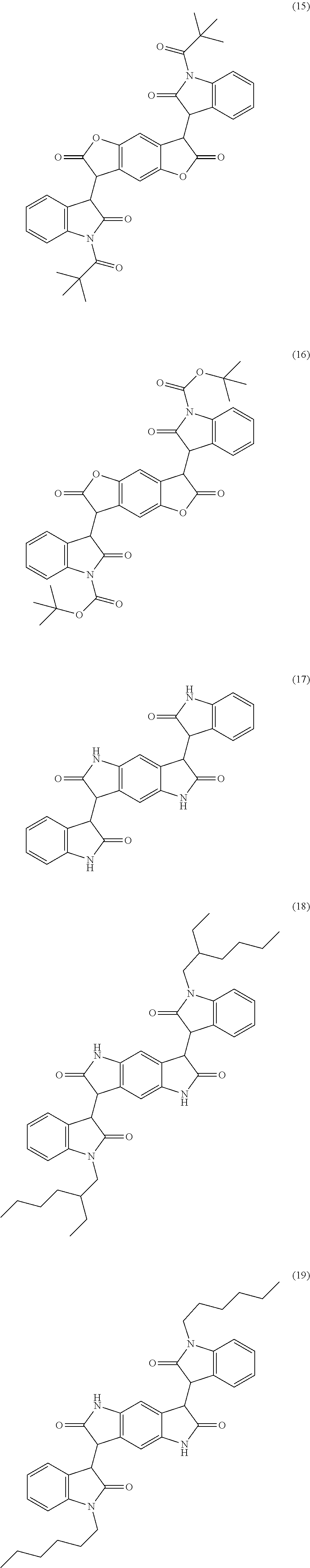

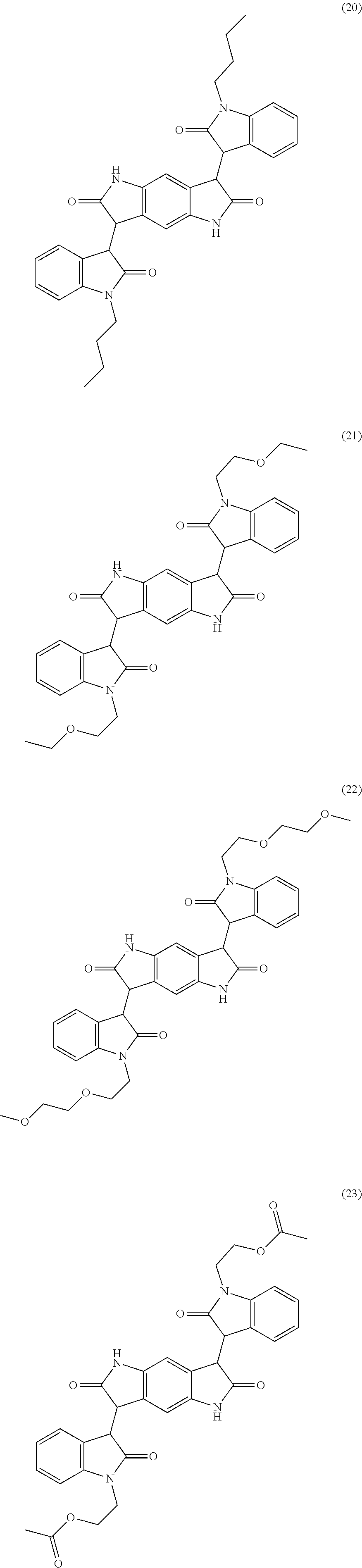

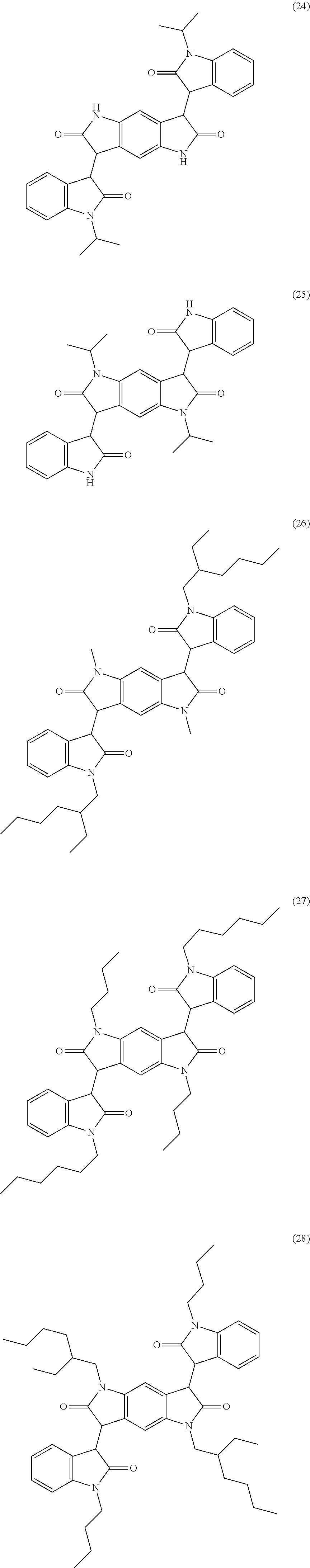

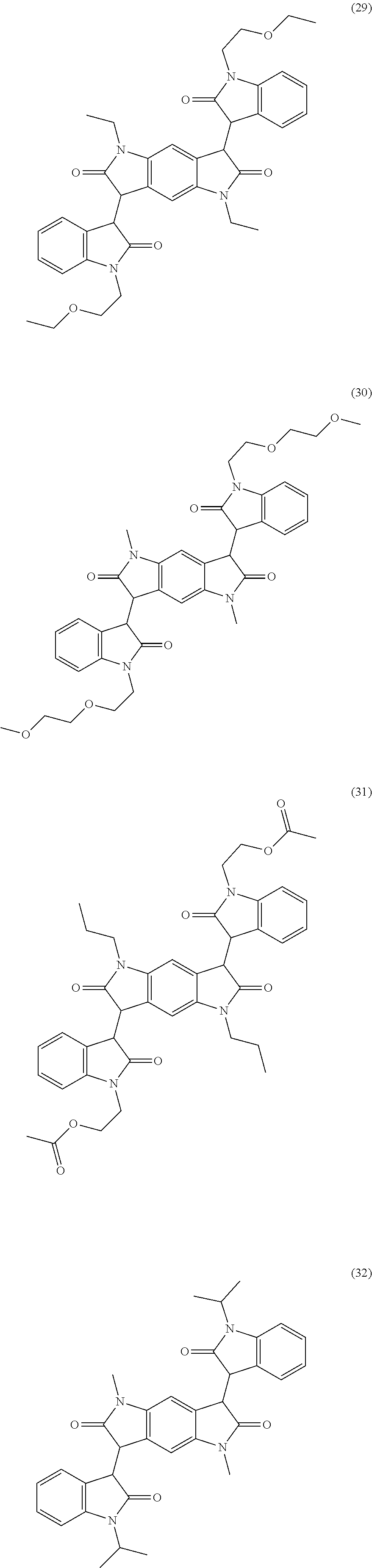

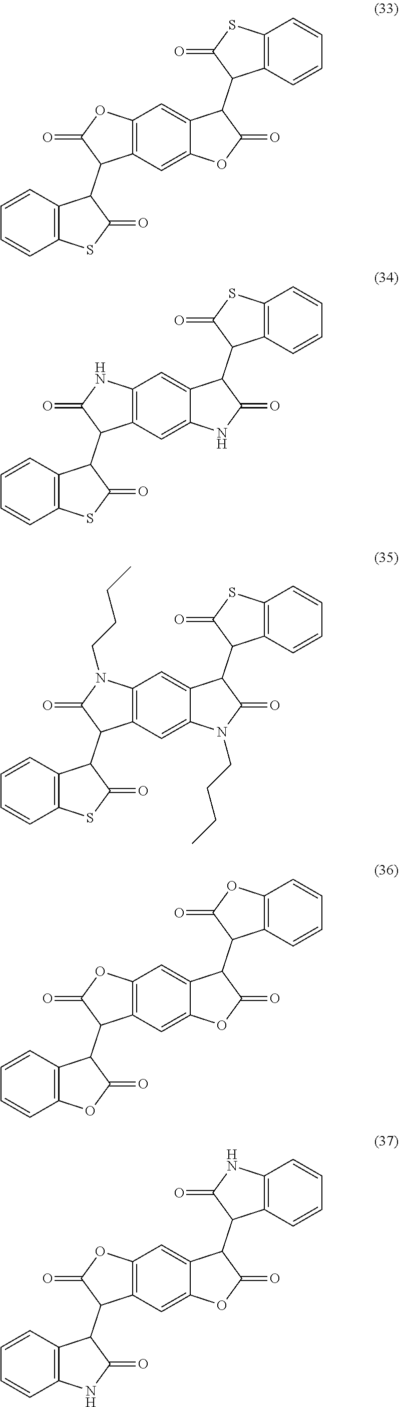 [SMILES: O=C1Oc2cc3c(cc2C1C1C(=O)Sc2ccccc21)OC(=O)C3C1C(=O)Sc2ccccc21 ; O=C1Nc2cc3c(cc2C1C1C(=O)Sc2ccccc21)NC(=O)C3C1C(=O)Sc2ccccc21 ; CCCCN1C(=O)C(C2C(=O)Sc3ccccc32)c2cc3c(cc21)C(C1C(=O)Sc2ccccc21)C(=O)N3CCCC ; O=C1Oc2ccccc2C1C1C(=O)Oc2cc3c(cc21)OC(=O)C3C1C(=O)Oc2ccccc21 ; O=C1Nc2ccccc2C1C1C(=O)Oc2cc3c(cc21)OC(=O)C3C1C(=O)Nc2ccccc21]

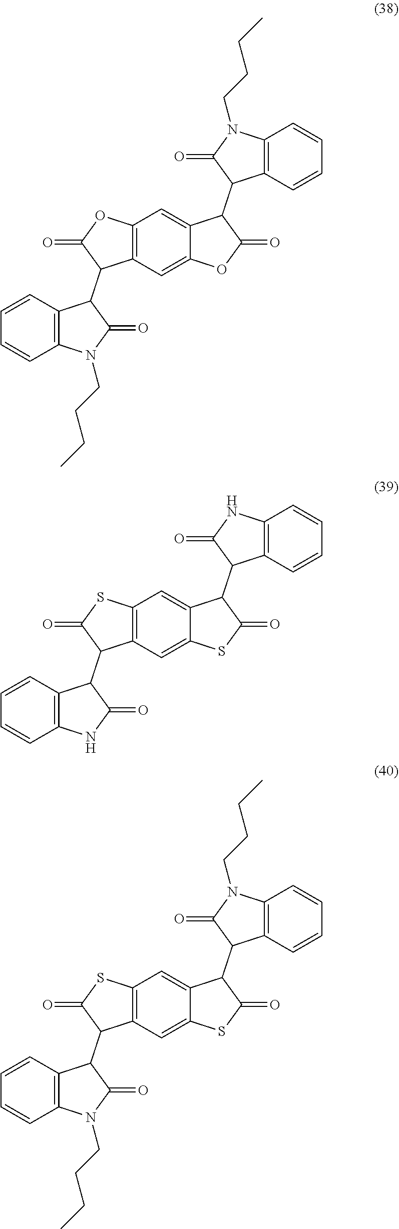 [SMILES: CCCCN1C(=O)C(C2C(=O)Oc3cc4c(cc32)OC(=O)C4C2C(=O)N(CCCC)c3ccccc32)c2ccccc21 ; O=C1Nc2ccccc2C1C1C(=O)Sc2cc3c(cc21)SC(=O)C3C1C(=O)Nc2ccccc21 ; CCCCN1C(=O)C(C2C(=O)Sc3cc4c(cc32)SC(=O)C4C2C(=O)N(CCCC)c3ccccc32)c2ccccc21]

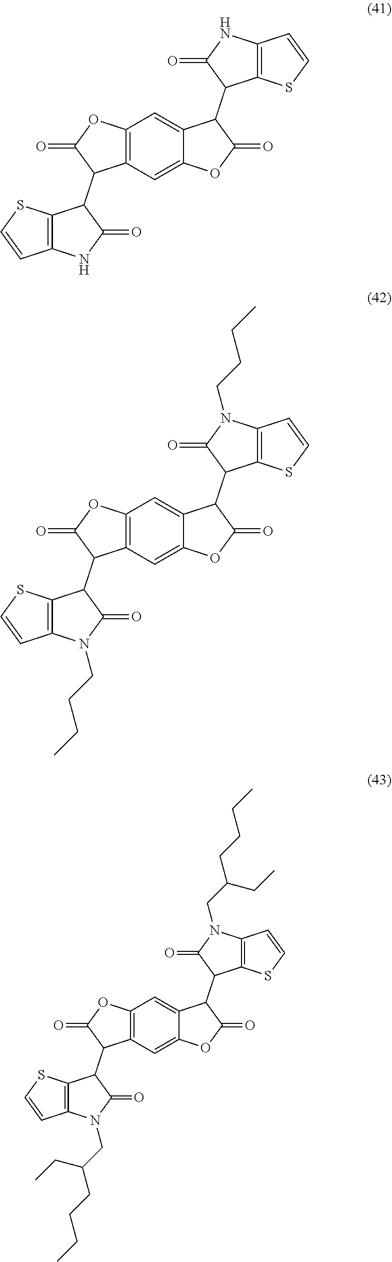 [SMILES: O=C1Oc2cc3c(cc2C1C1C(=O)Nc2ccsc21)OC(=O)C3C1C(=O)Nc2ccsc21 ; CCCCN1C(=O)C(C2C(=O)Oc3cc4c(cc32)OC(=O)C4C2C(=O)N(CCCC)c3ccsc32)c2sccc21 ; CCCCC(CC)CN1C(=O)C(C2C(=O)Oc3cc4c(cc32)OC(=O)C4C2C(=O)N(CC(CC)CCCC)c3ccsc32)c2sccc21]

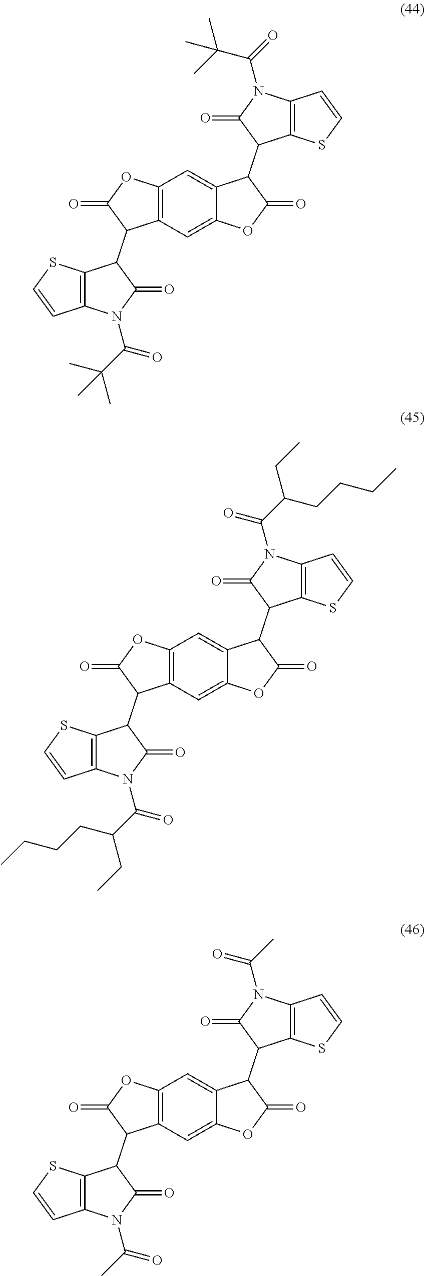 [SMILES: CC(C)(C)C(=O)N1C(=O)C(C2C(=O)Oc3cc4c(cc32)OC(=O)C4C2C(=O)N(C(=O)C(C)(C)C)c3ccsc32)c2sccc21 ; CCCCC(CC)C(=O)N1C(=O)C(C2C(=O)Oc3cc4c(cc32)OC(=O)C4C2C(=O)N(C(=O)C(CC)CCCC)c3ccsc32)c2sccc21 ; CC(=O)N1C(=O)C(C2C(=O)Oc3cc4c(cc32)OC(=O)C4C2C(=O)N(C(C)=O)c3ccsc32)c2sccc21]

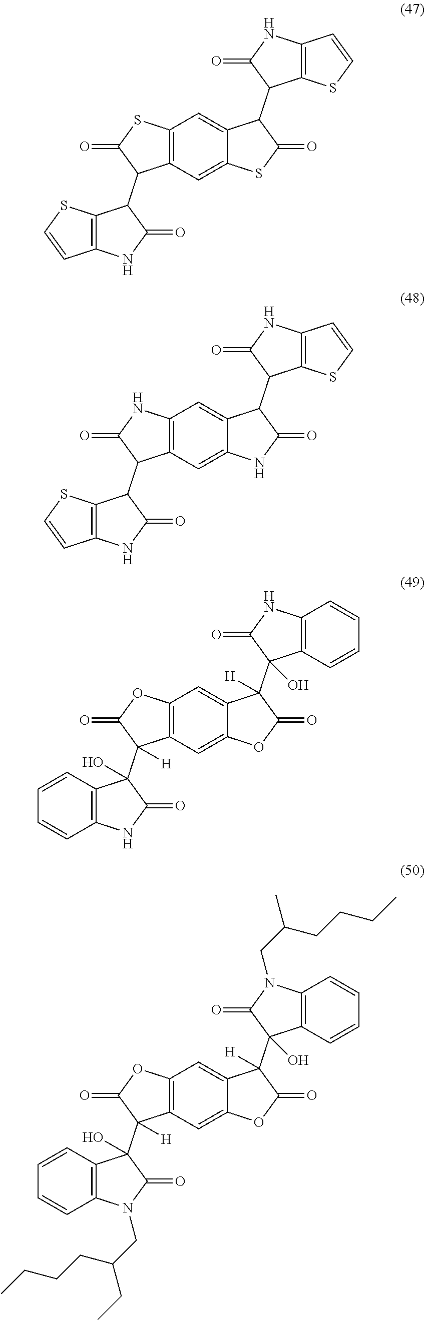 [SMILES: O=C1Sc2cc3c(cc2C1C1C(=O)Nc2ccsc21)SC(=O)C3C1C(=O)Nc2ccsc21 ; O=C1Nc2cc3c(cc2C1C1C(=O)Nc2ccsc21)NC(=O)C3C1C(=O)Nc2ccsc21 ; O=C1Oc2cc3c(cc2C1C1(O)C(=O)Nc2ccccc21)OC(=O)C3C1(O)C(=O)Nc2ccccc21 ; CCCCC(C)CN1C(=O)C(O)(C2C(=O)Oc3cc4c(cc32)OC(=O)C4C2(O)C(=O)N(CC(CC)CCCC)c3ccccc32)c2ccccc21]

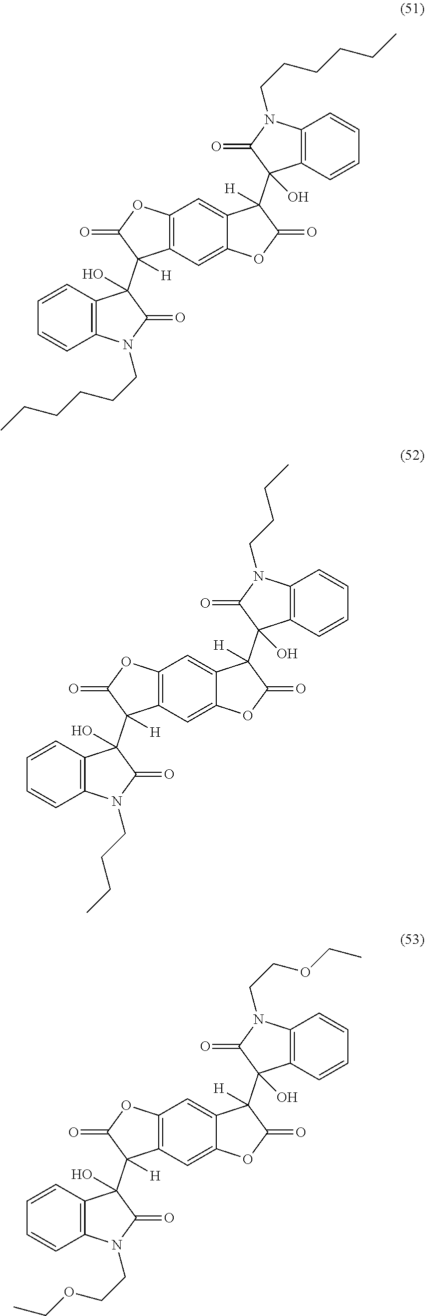

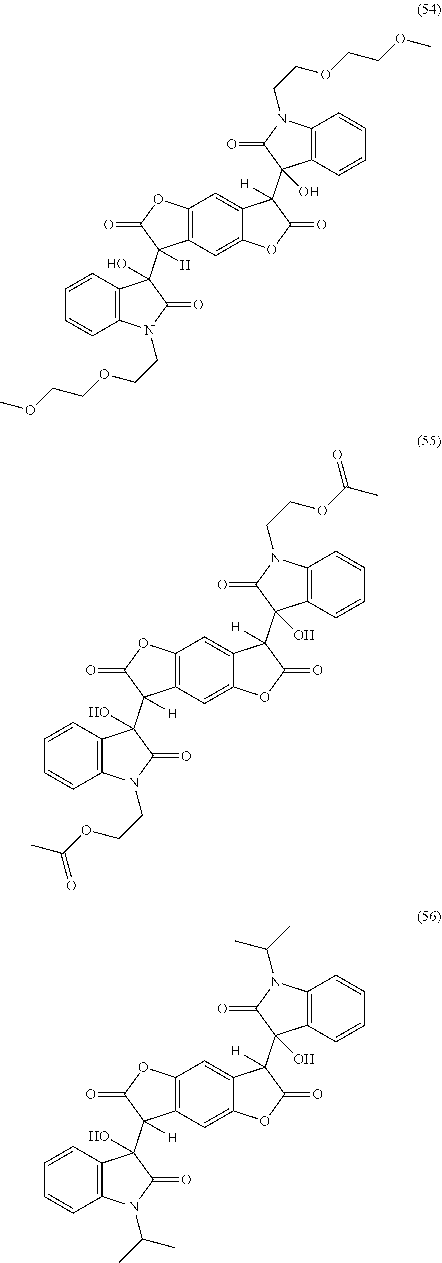

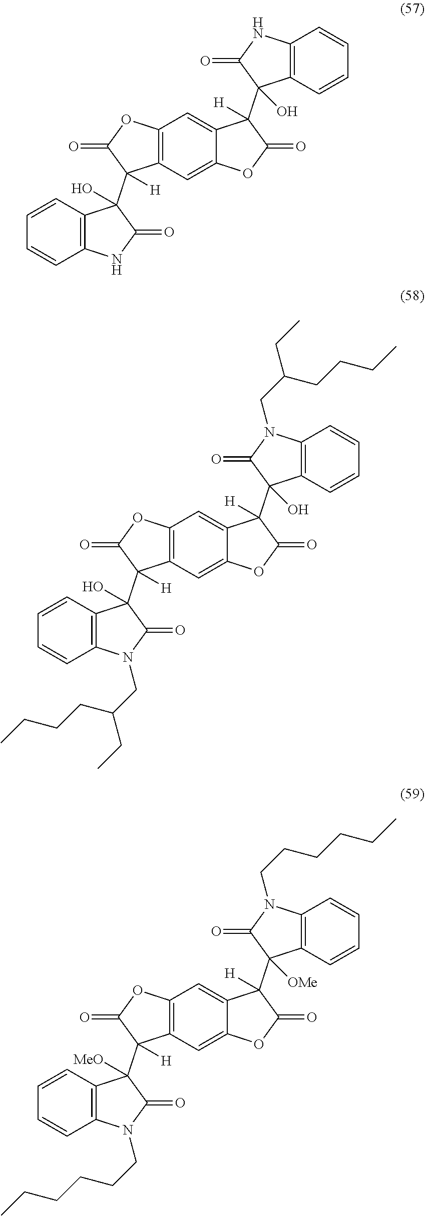

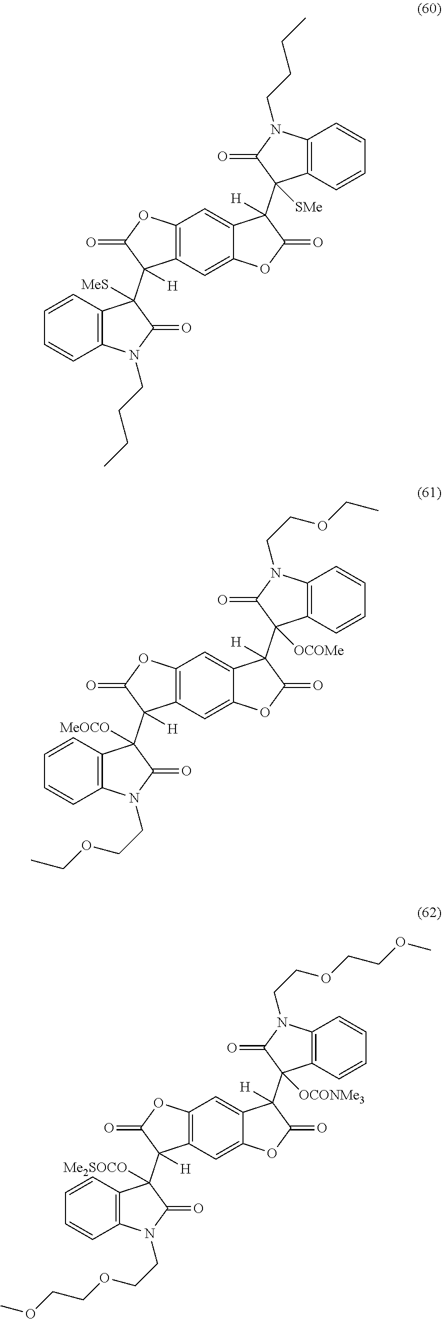

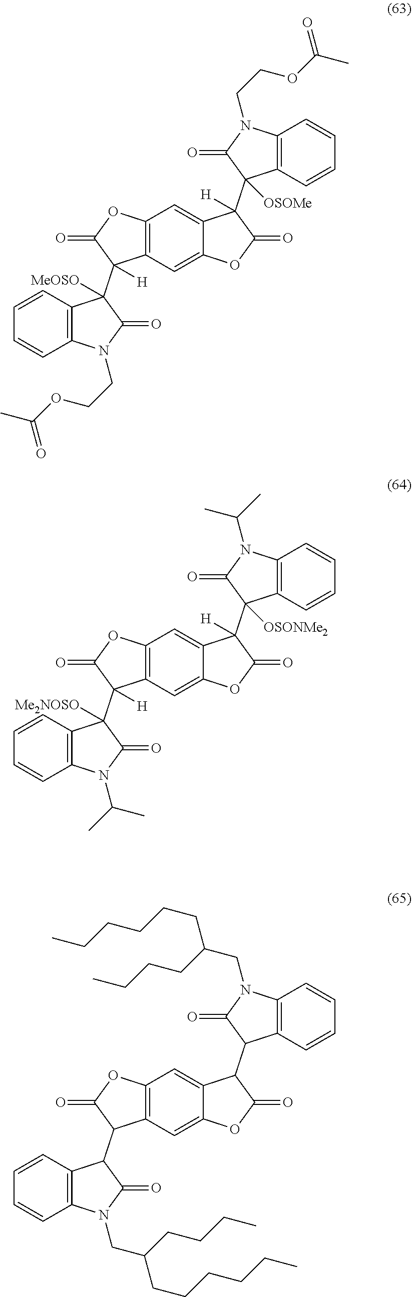

Among the above specific examples, as the compound represented by Formula (1), at least one selected from the group consisting of the compound (1) to the compound (16), the compound (25) to the compound (32), and the compound (65) is preferable; at least one selected from the group consisting of the compound (1) to the compound (16) and the compound (65) is more preferable; at least one selected from the group consisting of the compound (1) to the compound (3), the compound (5), the compound (7), and the compound (8) is still more preferable; and at least one selected from the group consisting of the compound (1) to the compound (3), the compound (5), the compound (7), and the compound (8) is particularly preferable.

The compound according to the present disclosure is colorless.

In the present disclosure, the expression that the compound is “colorless” means that a molar absorption coefficient of the compound in a solution state is less than 2,000 L/(mol·cm) in a wavelength range of 400 nm to 780 nm.

The molar absorption coefficient of the compound in a solution state is confirmed to be less than 2,000 L/(mol·cm) in a wavelength range of 400 nm to 780 nm by the following method. 1.1 mg of the compound is dissolved in 50 mL of tetrahydrofuran (THF). The obtained solution is placed in a 1 cm cell, and an absorption spectrum is measured using a spectrophotometer as a measuring device to obtain the molar absorption coefficient (F).

The compound according to the present disclosure has a characteristic of being colored by heating.

The above-described reaction in the compound according to the present disclosure is an irreversible reaction.

In the present disclosure, the state in which the compound is “colored” refers to a state in which a maximal absorption wavelength (λmax) of the compound in a solution state is in a wavelength range of 400 nm to 780 nm, and the molar absorption coefficient at the maximum absorption wavelength (λmax) is 2,000 L/(mol·cm) or more.

As an aspect of a colored compound in a case where the compound according to the present disclosure is colored by heating, an aspect in which a maximal absorption wavelength (λmax) of the compound in a solution state is in a wavelength range of 400 nm to 650 nm and the molar absorption coefficient at the maximal absorption wavelength (λmax) is 2,000 L/(mol·cm) or more is preferable. In the present disclosure, the fact that the molar absorption coefficient of the compound in a solution state is 2,000 L/(mol·cm) or more in the entire wavelength range of 400 nm to 650 nm means that the compound is in a state of being “colored black”.

Examples of a heating temperature for coloring the compound according to the present disclosure include 80° C. to 260° C.

### [Method for Producing Compound Represented by Formula (1)]

A method for producing the compound represented by Formula (1) (that is, the compound according to the present disclosure) is not particularly limited. The compound represented by Formula (1) can be produced with reference to a known method.

The compound represented by Formula (1) can be produced, for example, by synthesizing an isatin derivative using isatin as a starting material, reacting the synthesized isatin derivative with 3,7-Dihydrobenzo[1,2-b:4,5-b′]difuran-2,6-dione in an organic solvent under an acid catalyst, and reducing the compound obtained by the reaction, with reference to known documents.

Examples of the organic solvent include an ether-based organic solvent; and tetrahydrofuran (THF) and/or 1,4-dioxane is preferable, and tetrahydrofuran (THF) is more preferable.

Examples of a method of reducing the compound obtained by the reaction include a method using a reducing agent such as zinc powder, trifluoroacetic acid, acetic acid, and hydrochloric acid. In addition, the reduction may be contact reduction using a palladium catalyst. As the reduction method, reduction using zinc powder (so-called zinc reduction) or contact reduction using a palladium catalyst is preferable, and zinc reduction is more preferable.

A reaction temperature is not particularly limited, but is, for example, preferably 20° C. to 40° C. and more preferably 30° C. to 40° C.

A reaction time is not particularly limited, but is, for example, preferably 1 hour to 6 hours and more preferably 1 hour to 2 hours.

The method for synthesizing the isatin derivative is described in, for example, J. Am. Chem. Soc. 2015, 137, pp. 15947 to 15956, Journal of Medicinal Chemistry, 2008, 51, pp. 4932 to 4947, Chemistry-A European Journal, 2021, 27, pp. 4302 to 4306, Org. Lett., 2021, 23, pp. 2273 to 2278, and the like. The description of these documents is incorporated in the present specification by reference.

The compound represented by Formula (1) can be suitably produced by a method described in Examples later.

### <Applications>

The compound according to the present disclosure is a compound suitably used as a coloring agent precursor.

Since the compound according to the present disclosure has a characteristic of being colorless before heating and being colored after heating, the compound according to the present disclosure is suitable as a coloring agent precursor.

With the compound according to the present disclosure, since both transmission and shielding of light can be achieved, in an article (for example, a film or a molded body) containing the compound according to the present disclosure, the transmission and shielding of light can be controlled by heating.

Examples of specific applications of the compound according to the present disclosure include a black coloring material for a display element, specifically, a black coloring material for a black matrix (so-called black partition wall).

In a case where the compound according to the present disclosure is used as the black coloring material of a black matrix, for example, the compound is used as follows. A composition containing the compound according to the present disclosure, a resin, a polymerization initiator, a polymerizable compound, and the like is prepared, a coating film of the composition is formed, and then the coating film is exposed through a photo mask. Next, the exposed coating film is developed. Next, the developed coating film is post-baked, and the compound according to the present disclosure is reacted with oxygen by heating during the post-baking, thereby obtaining a black matrix which is a cured film colored black.

### [Composition]

The composition according to the present disclosure contains the compound represented by Formula (1) (that is, the compound according to the present disclosure). The composition according to the present disclosure includes not only a composition having a liquid form (so-called liquid composition), but also a composition having a solid form. Examples of the composition having a solid form include a membrane (for example, a film) and a molded body.

Since the composition according to the present disclosure contains the compound represented by Formula (1), the transmission and shielding of light can be controlled by heating. That is, since the compound represented by Formula (1) is colorless before heating, the composition according to the present disclosure can transmit light before heating; and since the compound represented by Formula (1) is colored (preferably, colored black) after heating, the composition according to the present disclosure can shield light after heating.

### <Compound Represented by Formula (1)>

Details of the compound represented by Formula (1) (that is, the compound according to the present disclosure) are as described above, and thus the description thereof will be omitted.

The composition according to the present disclosure may contain only one kind of the compound represented by Formula (1), or may contain two or more kinds thereof.

A content of the compound represented by Formula (1) in the composition according to the present disclosure is not particularly limited, but it is, for example, preferably 1% by mass to 60% by mass, more preferably 2% by mass to 55% by mass, and still more preferably 3% by mass to 50% by mass with respect to the total solid content mass of the composition.

### <Resin>

It is preferable that the composition according to the present disclosure further contains a resin.

In a case where the form of the composition according to the present disclosure is a solid (for example, a film or a molded body), the resin can function as a binder.

The resin preferably includes at least one of a thermoplastic resin or a thermosetting resin.

In the present disclosure, the thermoplastic resin means a resin which is softened and exhibits plasticity by heating, and is cured by cooling.

In the present disclosure, the thermosetting resin means a resin which is cured by heating. In addition, in the present disclosure, the thermosetting resin includes a resin in which a crosslinking structure or the like is formed by heating, which is partially or completely cured.

Examples of the thermoplastic resin include a polyolefin resin such as polyethylene, polypropylene, and an ethylene-propylene copolymer; a saturated polyester resin such as a (meth)acrylic resin and polyethylene terephthalate; an acrylonitrile styrene (AS) resin, an acrylonitrile butadiene styrene (ABS) resin, a polyvinylidene chloride resin, a polyamide resin, an acetal resin, a polycarbonate resin, a polyphenylene sulfide resin, a polyether imide resin, an aromatic polyether ketone resin, a polysulfone resin, a fluororesin such as polyvinylidene fluoride, a polyamide imide resin, and a polyether ether ketone (PEEK) resin.

A melting point of the thermoplastic resin is not particularly limited, but for example, from the viewpoint of heat resistance of the film or the molded body, it is preferably 100° C. or higher, more preferably 110° C. or higher, and still more preferably 130° C. or higher.

The upper limit of the melting point of the thermoplastic resin is not particularly limited, but is, for example, preferably lower than 230° C. and more preferably 180° C. or lower.

The compound represented by Formula (1) is colored by reacting with oxygen in a case of being heated to, for example, 230° C. or higher. In a case where the melting point of the thermoplastic resin is lower than 230° C., the reaction of the compound represented by Formula (1) with oxygen due to heat in a case of melting the thermoplastic resin is suppressed, and thus the composition according to the present disclosure tends to be less colored.

From such a viewpoint, the thermoplastic resin is preferably a resin which is melted at a low temperature, such as polyethylene and polypropylene.

The melting point of the thermoplastic resin is measured in accordance with JIS K 7121:2012 (ISO 3146:1985) using a differential scanning calorimeter as a measuring device by raising the temperature of the thermoplastic resin from 30° C. to 600° C. at a temperature rising rate of 10° C./min.

As the differential scanning calorimeter, for example, a differential scanning calorimeter (model number: DSC7000X) manufactured by Hitachi High-Tech Science Corporation can be used.

Examples of the thermosetting resin include an epoxy resin, a phenoxy resin, a phenol resin, a polystyrene resin, a urea resin, a melamine resin, an unsaturated polyester resin, a diallyl phthalate resin, a polyurethane resin, a silicon resin, a polyimide resin, a polyisoprene rubber, a polybutadiene rubber, a styrene-butadiene copolymer rubber, a butyl rubber, an acrylonitrile-butadiene rubber, a chloroprene rubber, and a silicone rubber.

With regard to the details of the resin, reference can be made to, for example, description in paragraphs [0075] to [0097] of JP2009-263616A, the contents of which are incorporated herein by reference.

The resin may be an alkali-soluble resin.

In the present disclosure, the “alkali-soluble” refers to being soluble in a 1 mol/L sodium hydroxide solution at 25° C. In addition, the “soluble” refers to being dissolved in an amount of 0.1 g or more in 100 mL of a solvent.

The alkali-soluble resin is preferably a resin having a group which promotes alkali solubility (hereinafter, also referred to as “acid group”).

Examples of the acid group include a carboxy group, a phosphoric acid group, a sulfonic acid group, and a phenolic hydroxy group.

Among these, as the acid group, a carboxy group is preferable.

In a case where the resin has an acid group, the resin may have only one kind of the acid group or may have two or more kinds thereof.

The alkali-soluble resin is preferably a polymer having a carboxy group in a side chain.

Examples of the alkali-soluble resin include (meth)acrylic acid copolymers, itaconic acid copolymers, crotonic acid copolymers, maleic acid copolymers, partially esterified maleic acid copolymers, alkali-soluble phenol resins such as a novolac-type resin, acidic cellulose derivatives having a carboxy group in the side chain, and resins in which an acid anhydride is added to a polymer having a hydroxy group.

The alkali-soluble resin is preferably a copolymer of (meth)acrylic acid and other monomers copolymerizable with the (meth)acrylic acid [so-called (meth)acrylic acid copolymer].

Examples of the other monomers copolymerizable with the (meth)acrylic acid include alkyl (meth)acrylate, aryl (meth)acrylate, and a vinyl compound.

Specific examples of the other monomers copolymerizable with the (meth)acrylic acid include methyl (meth)acrylate, ethyl (meth)acrylate, propyl (meth)acrylate, butyl (meth)acrylate, isobutyl (meth)acrylate, pentyl (meth)acrylate, hexyl (meth)acrylate, octyl (meth)acrylate, phenyl (meth)acrylate, benzyl (meth)acrylate, tolyl (meth)acrylate, naphthyl (meth)acrylate, cyclohexyl (meth)acrylate, styrene, a-methylstyrene, vinyl toluene, glycidyl methacrylate, acrylonitrile, vinyl acetate, N-vinylpyrrolidone, tetrahydrofurfuryl methacrylate, a polystyrene macromonomer, and a polymethyl methacrylate macromonomer.

Examples of the other monomers copolymerizable with the (meth)acrylic acid also include N-position-substituted maleimides described in JP1998-300922A (JP-H10-300922A) (for example, N-phenylmaleimide and N-cyclohexylmaleimide).

In the (meth)acrylic acid copolymer, the other monomers copolymerizable with the (meth)acrylic acid may be only one kind or may be two or more kinds.

Specific examples of the alkali-soluble resin also include a benzyl (meth)acrylate/(meth)acrylic acid copolymer, a benzyl (meth)acrylate/(meth)acrylic acid/2-hydroxyethyl (meth)acrylate copolymer, and a multicomponent copolymer formed of benzyl (meth)acrylate/(meth)acrylic acid/other monomers.

In addition, preferred examples of the alkali-soluble resin also include resins obtained by copolymerizing 2-hydroxyethyl (meth)acrylate, and a copolymer of 2-hydroxypropyl (meth)acrylate/polystyrene macromonomer/benzyl methacrylate/methacrylic acid, a copolymer of 2-hydroxy-3-phenoxypropyl acrylate/polymethyl methacrylate macromonomer/benzyl methacrylate/methacrylic acid, a copolymer of 2-hydroxyethyl methacrylate/polystyrene macromonomer/methyl methacrylate/methacrylic acid, and a copolymer of 2-hydroxyethyl methacrylate/polystyrene macromonomer/benzyl methacrylate/methacrylic acid, which are described in JP1995-140654A (JP-H7-140654A).

With regard to the details of the alkali-soluble resin, reference can be made to, for example, description in JP2011-225806A, JP2012-208494A, and JP2012-198408A, the contents of which are incorporated herein by reference.

An acid value of the alkali-soluble resin is not particularly limited, but is, for example, preferably 30 mgKOH/g to 200 mgKOH/g, more preferably 50 mgKOH/g to 150 mgKOH/g, and still more preferably 70 mgKOH/g to 120 mgKOH/g.

In the present disclosure, the acid value is a value measured by a method described in JIS K 0070: 1992.

The resin may have a polymerizable group.

In a case where the composition according to the present disclosure contains the resin having a polymerizable group, it is possible to form, for example, a film, a molded body, or the like having higher hardness.

Specific examples of the polymerizable group include a vinyl group, a (meth)allyl group, a (meth)acryloyl group, a (meth)acryloyloxy group, a (meth)acryloylamino group, and a vinylphenyl group.

A weight-average molecular weight of the resin is not particularly limited, but for example, preferably 1,000 to 200,000, more preferably 2,000 to 200,000, and still more preferably 5,000 to 50,000.

In a case where the composition according to the present disclosure contains a resin, the composition according to the present disclosure may contain only one kind of the resin, or may contain two or more kinds thereof.

In a case where the composition according to the present disclosure contains a resin, a content of the resin is not particularly limited, but for example, preferably 10% by mass to 90% by mass, more preferably 20% by mass to 80% by mass, and still more preferably 30% by mass to 70% by mass with respect to the total solid content mass of the composition.

### <Other Components>

The composition according to the present disclosure may contain a component other than the above-described components (so-called other components) as necessary, within a range in which the effect of the present disclosure is not impaired.

Examples of the other components include various additives.

Examples of the additive include a crosslinking agent such as sulfur, a polymerization initiator, an antioxidant, a lubricant such as paraffin oil, a plasticizer, a preservative, a fungicide, and an antistatic agent.

Examples of the sulfur include powder sulfur, precipitated sulfur, colloidal sulfur, insoluble sulfur, highly dispersible sulfur, and soluble sulfur. These sulfurs may be used alone or in combination of two or more kinds thereof.

### [Film]

The film according to the present disclosure contains the compound represented by Formula (1) (that is, the compound according to the present disclosure). The film according to the present disclosure is also one aspect of the composition according to the present disclosure.

Since the film according to the present disclosure contains the compound represented by Formula (1), the transmission and shielding of light can be controlled by heating. That is, since the compound represented by Formula (1) is colorless before heating, the film according to the present disclosure can transmit light before heating; and since the compound represented by Formula (1) is colored (preferably, colored black) after heating, the film according to the present disclosure can shield light after heating.

For example, the film according to the present disclosure can transmit light in a wide wavelength range from ultraviolet rays in a wavelength range longer than 350 nm to visible rays and infrared rays.

### <Compound Represented by Formula (1)>

Details of the compound represented by Formula (1) (that is, the compound according to the present disclosure) are as described above, and thus the description thereof will be omitted.

The film according to the present disclosure may contain only one kind of the compound represented by Formula (1), or may contain two or more kinds thereof.

A content of the compound represented by Formula (1) in the film according to the present disclosure is not particularly limited, but it is, for example, preferably 1% by mass to 60% by mass, more preferably 5% by mass to 55% by mass, and still more preferably 10% by mass to 50% by mass with respect to the total mass of the film.

### <Resin>

The film according to the present disclosure preferably contains a resin, in addition to the compound represented by Formula (1). In the film according to the present disclosure, the resin can function as a binder.

Details of the resin are the same as those of the resin contained in the composition according to the present disclosure described above, and thus the description thereof will be omitted.

In a case where the film according to the present disclosure contains a resin, the film according to the present disclosure may contain only one kind of the resin, or may contain two or more kinds thereof.

In a case where the film according to the present disclosure contains a resin, a content of the resin is not particularly limited, but for example, preferably 1% by mass to 60% by mass, more preferably 5% by mass to 55% by mass, and still more preferably 10% by mass to 50% by mass with respect to the total mass of the film.

### <Other Components>

The film according to the present disclosure may contain a component other than the above-described components (so-called other components) as necessary, within a range in which the effect of the present disclosure is not impaired.

Examples of the other components include various additives.

Examples of the additive include a colorant, a preservative, an antibacterial agent, and an antistatic agent.

A film thickness of the film according to the present disclosure is not particularly limited, but for example, preferably 5 μm to 100 μm, more preferably 10 μm to 50 μm, and still more preferably 20 μm to 30 μm.

### [Method for Producing Coloring Composition]

The method for producing a coloring composition according to the present disclosure includes a step A of obtaining a composition containing the compound represented by Formula (1) (that is, the compound according to the present disclosure) and a resin, and a step B of heating the composition.

Hereinafter, the “composition containing the compound represented by Formula (1) and a resin” in the method for producing a coloring composition according to the present disclosure will also be referred to as “resin composition”.

### <Step A>

The step A is a step of obtaining a composition (that is, a resin composition) containing the compound represented by Formula (1) and a resin. First, a method of obtaining a resin composition will be described, and then components contained in or capable of being contained in the resin composition will be described.

The method of obtaining the resin composition varies depending on a form of the resin composition. Hereinafter, specific examples of the method of obtaining the resin composition will be described for each form of the resin composition. However, the method of obtaining the resin composition is not limited to the following methods.

In a case where the resin composition is a liquid composition, examples of the method of obtaining the resin composition include a method of obtaining the resin composition by mixing or kneading at least the compound represented by Formula (1) and the resin.

The mixing or kneading method is not particularly limited.

All components blended in the resin composition may be mixed or kneaded at once, or each component blended in the resin composition may be mixed or kneaded in several times.

The components blended in the resin composition may be simply mixed or kneaded in the resin composition, but are preferably uniformly mixed or kneaded.

The mixing can be carried out using a general stirring instrument or a stirring device.

The kneading can be carried out using a general kneading device.

The mixing temperature or kneading temperature is not particularly limited and can be appropriately set depending on the type, formulation, and the like of the resin to be mixed or kneaded.

The mixing time or kneading time is not particularly limited and can be appropriately set depending on the type of the instrument or device used for the mixing or kneading, the formulation of the resin composition, and the like.

In a case where the resin composition is a membrane (for example, a film), examples of the method of obtaining the resin composition include a method of obtaining the resin composition by mixing or kneading at least the compound represented by Formula (1) and the resin, forming a mixture layer or a kneaded layer using the obtained mixture or kneaded material, and curing the mixture layer or the kneaded layer by applying energy to the formed mixture layer or kneaded layer.

The mixture layer or the kneaded layer may be formed on a desired support.

The method of applying energy to the mixture layer or the kneaded layer is not particularly limited, and examples thereof include heating and light irradiation. In a case where the heating is selected as the method of applying energy, from the viewpoint of suppressing the compound represented by Formula (1) from being colored by heating, it is preferable to carry out heating at a temperature lower than 230° C. The method of applying energy is preferably light irradiation, and more preferably irradiation with ultraviolet rays.

In a case where the light irradiation is adopted as the application of the energy, it is preferable that the resin composition contains a photopolymerization initiator described later.

In a case where the mixture layer or the kneaded layer is cured by irradiation with ultraviolet rays, for example, an ultraviolet lamp (for example, a high-pressure mercury lamp) can be used for the irradiation with ultraviolet rays.

A light irradiation amount of ultraviolet rays is not particularly limited, but for example, preferably 10 mJ/cm² to 1,000 mJ/cm². In a case where the light irradiation amount of ultraviolet rays is within the above-described range, the mixture layer or the kneaded layer tends to be cured more suitably.

From the viewpoint of further promoting the curing reaction of the mixture layer or the kneaded layer, a temperature at which the mixture layer or the kneaded layer is cured is, for example, preferably 25° C. to 100° C., more preferably 30° C. to 80° C., and still more preferably 40° C. to 70° C.

In a case where the resin composition contains a solvent, it is preferable to dry the mixture layer or the kneaded layer in advance to reduce the amount of the solvent before applying energy to the mixture layer or the kneaded layer, from the viewpoint of improving curing properties of the mixture layer or the kneaded layer.

The method of drying the mixture layer or the kneaded layer is not particularly limited, and a known drying method can be adopted.

Examples of the drying method of the mixture layer or the kneaded layer include a method of blowing hot air onto the mixture layer or the kneaded layer, a method of passing the mixture layer or the kneaded layer through a drying zone controlled at a predetermined temperature, and a method of transporting the mixture layer or the kneaded layer by a transport roll equipped with a heater.

In a case where the resin composition is a molded body, examples of the method of obtaining the resin composition include a method of, for example, kneading at least the compound represented by Formula (1) and the resin, and then molding the obtained kneaded material.

In a case where the resin is a thermoplastic resin, the resin may be heated before kneading with the compound represented by Formula (1), and then the compound represented by Formula (1) and the molten resin may be kneaded; or the compound represented by Formula (1) and the resin may be kneaded while being heated in a case where the compound represented by Formula (1) and the resin are kneaded.

A temperature at which the resin is heated can be appropriately adjusted according to a melting point of the thermoplastic resin, but from the viewpoint of avoiding coloration of the resin composition before the heating in the step B, for example, it is preferably lower than 230° C., and more preferably 80° C. or higher and lower than 230° C.

The method of molding the kneaded material is not particularly limited, and a known molding method can be adopted.

Examples of the method of molding the kneaded material include injection molding, extrusion molding, and press molding.

It is preferable that a molding temperature of the kneaded material is appropriately set, for example, depending on the type of the resin, but from the viewpoint of avoiding the coloration of the resin composition to black before the heating in the step B, it is, for example, preferably lower than 230° C., and more preferably 80° C. or higher and lower than 230° C.

In addition, in a case where the resin composition is a molded body, examples of the method of obtaining the resin composition also include a method of obtaining the resin composition by mixing or kneading at least the compound represented by Formula (1) and the resin, filling a desired molding mold with the obtained mixture or kneaded material, and curing the mixture layer or the kneaded layer by applying energy to the formed mixture layer or kneaded layer.

Components contained in or capable of being contained in the resin composition will be described.

### (Compound Represented by Formula (1))

Details of the compound represented by Formula (1) (that is, the compound according to the present disclosure) are as described above, and thus the description thereof will be omitted.

The resin composition may contain only one kind of the compound represented by Formula (1), or may contain two or more kinds thereof.

A content of the compound represented by Formula (1) in the resin composition is not particularly limited, but it is, for example, preferably 1% by mass to 60% by mass, more preferably 2% by mass to 55% by mass, and still more preferably 3% by mass to 50% by mass with respect to the total solid content mass of the resin composition.

### (Resin)

Details of the resin are the same as those of the resin contained in the composition according to the present disclosure described above, and thus the description thereof will be omitted.

The resin composition may contain only one kind of the resin or two or more kinds thereof.

A content of the resin in the resin composition is not particularly limited, but for example, preferably 1% by mass to 90% by mass, more preferably 5% by mass to 80% by mass, and still more preferably 10% by mass to 70% by mass with respect to the total solid content mass of the resin composition.

The resin composition may further contain a polymerization initiator and a polymerizable monomer.

The resin composition can be used as a negative-tone resin composition by containing the polymerization initiator and the polymerizable monomer, in addition to the compound represented by Formula (1) and the resin.

### (Polymerization Initiator)

The polymerization initiator may be any compound capable of generating an initiating species required for the polymerization reaction by application of energy, and is not particularly limited. As the polymerization initiator, a known polymerization initiator can be used.

Examples of the polymerization initiator include a photopolymerization initiator and a thermal polymerization initiator.

As the photopolymerization initiator, an activator having photosensitivity to light in a range from an ultraviolet range to a visible range is preferable. In addition, the photopolymerization initiator may be an activator which produces an active radical by causing some action with a photoexcited sensitizer.

Examples of the photopolymerization initiator include a halogenated hydrocarbon derivative (for example, a compound having a triazine skeleton and a compound having an oxadiazole skeleton), an acylphosphine compound, hexaaryl biimidazole, an oxime compound (for example, an oxime ester compound), an organic peroxide, a thio compound, a ketone compound, an aromatic onium salt, an aminoacetophenone compound, and a hydroxyacetophenone compound.

Examples of the acylphosphine compound include acylphosphine-based initiators described in JP4225898B.

Examples of the oxime compound include compounds described in JP2001-233842A, compounds described in JP2000-080068A, compounds described in JP2006-342166A, and compounds described in paragraphs [0073] to [0075] of JP2016-006475A. Among the oxime compounds, an oxime ester compound is preferable.

Examples of the aminoacetophenone compound include compounds described in JP2009-191179A and aminoacetophenone-based initiators described in JP1998-291969A (JP-H10-291969A).

In a case where the compound represented by Formula (1) is heated to 230° C. or higher, the compound is colored, so that, in a case where the polymerization initiator is a thermal polymerization initiator, the thermal polymerization initiator is preferably a compound capable of generating an initiating species necessary for a polymerization reaction by heat of, for example, lower than 230° C.

Examples of the thermal polymerization initiator include an azo-based compound, an organic peroxide, and an inorganic peroxide.

Specific examples of the azo-based compound include dimethyl 2,2′-azobis(isobutyrate), 2,2′-azobisisobutyronitrile, 2,2′-azobis(2,4-dimethyl-4-methoxyvaleronitrile), 2,2′-azobis(2,4-dimethylvaleronitrile), dimethyl-2,2′-azobis(2-methylpropionate), 2,2′-azobis(2-methylbutyronitrile), 1,1′-azobis(cyclohexane-1-carbonitrile), 2,2′-azobis(N-butyl-2-methylpropionamide), dimethyl-1,1′-azobis(1-cyclohexanecarboxylate), and 2,2′-azobis[2-(2-imidazolin-2-yl)propane]dihydrochloride.

Specific examples of the organic peroxide include 1,1-di(tert-hexylperoxy)cyclohexane, 1,1-di(tert-butylperoxy)cyclohexane, 2,2-di(4,4-di-(tert-butylperoxy)cyclohexyl)propane, tert-hexyl peroxy isopropyl monocarbonate, tert-butyl peroxy-3,5,5-trimethyl hexanoate, tert-butyl peroxy laurate, dicumyl peroxide, di-tert-butyl peroxide, tert-butyl peroxy-2-ethyl hexanoate, tert-hexyl peroxy-2-ethyl hexanoate, cumene hydroperoxide, and tert-butyl hydroperoxide.

Specific examples of the inorganic peroxide include potassium persulfate, ammonium persulfate, and hydrogen peroxide.

The polymerization initiator may be a synthetic product or a commercially available product.

Examples of the commercially available product of the photopolymerization initiator include IRGACURE (registered trademark) OXE01 [manufactured by BASF SE], TR-PBG-304 [manufactured by Changzhou Tronly New Electronic Materials Co., Ltd.], and ADEKA ARKLS (registered trademark) NCI-831 and ADEKA ARKLS (registered trademark) NCI-930 [both manufactured by ADEKA Corporation].

In addition, examples of the commercially available product of the photopolymerization initiator, which is the hydroxyacetophenone compound, include Omnirad (registered trademark) 184, Omnirad (registered trademark) 1173, Omnirad (registered trademark) 2959, and Omnirad (registered trademark) 127 [all manufactured by IGM Resins B.V.].

Examples of the commercially available product of the photopolymerization initiator, which is the aminoacetophenone compound, include Omnirad (registered trademark) 907, Omnirad (registered trademark) 369, Omnirad (registered trademark) 369E, and Omnirad (registered trademark) 379EG [all manufactured by IGM Resins B.V.].

Examples of the commercially available product of the photopolymerization initiator, which is the acylphosphine compound, include Omnirad (registered trademark) 819 and Omnirad (registered trademark) TPO [both manufactured by IGM Resins B.V.].

Examples of the commercially available product of the photopolymerization initiator, which is the oxime compound, include Irgacure (registered trademark) OXE01, Irgacure (registered trademark) OXE02, and Irgacure (registered trademark) OXE03 [all manufactured by BASF SE].

In a case where the resin composition contains a polymerization initiator, the resin composition may contain only one kind of the polymerization initiator, or may contain two or more kinds thereof.

In a case where the resin composition contains a polymerization initiator, a content of the polymerization initiator is not particularly limited, but for example, preferably 0.1% by mass to 20% by mass, more preferably 0.2% by mass to 15% by mass, and still more preferably 0.3% by mass to 10% by mass with respect to the total solid content mass of the resin composition.

### (Polymerizable Monomer)

The polymerizable monomer may be any compound which can be polymerized and cured by application of energy, and is not particularly limited. As the polymerizable monomer, a known polymerizable monomer can be used.

The polymerizable monomer is preferably a monomer having a polymerizable group.

The polymerizable group is preferably a group having an ethylenically unsaturated bond.

Specific examples of the polymerizable group include a vinyl group, a (meth)allyl group, a (meth)acryloyl group, a (meth)acryloyloxy group, a (meth)acryloylamino group, and a vinylphenyl group.

The polymerizable monomer is preferably a monomer having one or more terminal ethylenically unsaturated bonds.

Examples of the polymerizable monomer include an unsaturated carboxylic acid, an ester of an unsaturated carboxylic acid, and an amide of an unsaturated carboxylic acid.

Specific examples of the unsaturated carboxylic acid include (meth)acrylic acid, itaconic acid, crotonic acid, isocrotonic acid, and maleic acid.

It is preferable that the polymerizable monomer is at least one selected from the group consisting of a (meth)acrylic acid, a (meth)acrylic acid amide compound, a (meth)acrylic acid ester compound, and a styrene compound.

Specific examples of the (meth)acrylic acid amide compound include (meth)acrylic acid amide, N,N-dimethyl acrylamide, N-isopropyl acrylamide, methylenebis(acrylamide), 2-acrylamido-2-methylpropanesulfonic acid, and N-(3-dimethylaminopropyl)methacrylamide.

Specific examples of the (meth)acrylic acid ester compound include methyl (meth)acrylate, ethyl (meth)acrylate, n-butyl (meth)acrylate, 2-hydroxyethyl (meth)acrylate, glycidyl (meth)acrylate, benzyl (meth)acrylate, 2-(2-phenoxy)ethyl (meth)acrylate, 2-ethylhexyl (meth)acrylate, n-octyl (meth)acrylate, isooctyl (meth)acrylate, n-nonyl (meth)acrylate, isononyl (meth)acrylate, n-decyl (meth)acrylate, isodecyl (meth)acrylate, n-dodecyl (meth)acrylate, n-tridecyl (meth)acrylate, n-tetradecyl (meth)acrylate, n-hexadecyl (meth)acrylate, stearyl (meth)acrylate, isobornyl (meth)acrylate, dicyclopentenyl (meth)acrylate, dicyclopentanyl (meth)acrylate, dicyclopentenyloxyethyl (meth)acrylate, 1-hydroxyheptyl (meth)acrylate, 1-hydroxybutyl (meth)acrylate, 1-hydroxypentyl (meth)acrylate, 2-hydroxybutyl (meth)acrylate, ethylene glycol diacrylate, ethylene glycol dimethacrylate, polyethylene glycol diacrylate, polyethylene glycol dimethacrylate, ethoxylated bisphenol A diacrylate, ethoxylated bisphenol A dimethacrylate, ethoxylated trimethylolpropane triacrylate, ethoxylated trimethylolpropane trimethacrylate, ethoxylated glycerin triacrylate, ethoxylated glycerin trimethacrylate, ethoxylated pentaerythritol tetraacrylate, ethoxylated pentaerythritol tetramethacrylate, ethoxylated dipentaerythritol hexaacrylate, polyglycerin monoethylene oxide polyacrylate, polyglycerin polyethylene glycol polyacrylate, dipentaerythritol hexaacrylate, dipentaerythritol hexamethacrylate, neopentyl glycol diacrylate, neopentyl glycol dimethacrylate, pentaerythritol triacrylate, pentaerythritol trimethacrylate, trimethylolpropane triacrylate, trimethylolpropane trimethacrylate, tricyclodecane dimethanol diacrylate, tricyclodecane dimethanol dimethacrylate, 1,6-hexanediol diacrylate, and 1,6-hexanediol dimethacrylate.

Specific examples of the styrene compound include styrene, methylstyrene, dimethylstyrene, trimethylstyrene, ethylstyrene, fluorostyrene, chlorostyrene, methoxystyrene, tert-butoxystyrene, and divinylbenzene.

Regarding the polymerizable monomer, details of the usage method, such as what kind of polymerizable monomer having what kind of structure is used, whether the polymerizable monomer is used alone or in combination of two or more kinds thereof, and how much the content thereof is set, can be optionally set according to the final performance design of the resin composition.

For example, from the viewpoint of sensitivity, as the polymerizable monomer, a monomer having a structure in which the amount of polymerizable groups per molecule is large is preferable, and in many cases, a bi- or higher functional monomer is preferable.

For example, from the viewpoint of film hardness, as the polymerizable monomer, a tri- or higher functional compound [for example, a hexafunctional (meth)acrylic acid ester compound] may be used.

For example, it is also effective to select a polymerizable monomer in consideration of compatibility, dispersibility, and the like with each component contained in the resin composition.

As the polymerizable monomer, monomers having different functionalities and/or different polymerizable groups [for example, the (meth)acrylic acid ester compound, the styrene compound, and the vinyl ether compound] may be used in combination.

A molecular weight of the polymerizable monomer is not particularly limited, but is, for example, preferably 100 or more and less than 1,000, and more preferably 150 or more and less than 1,000.

In a case where the resin composition contains a polymerizable monomer, the resin composition may contain only one kind of the polymerizable monomer, or may contain two or more kinds thereof.

In a case where the resin composition contains a polymerizable monomer, a content of the polymerizable monomer is not particularly limited, but for example, preferably 5% by mass to 60% by mass, more preferably 10% by mass to 50% by mass, and still more preferably 20% by mass to 40% by mass with respect to the total solid content mass of the resin composition.

### (Polymerization Inhibitor)

In a case where the resin composition is used as a negative-tone resin composition, the resin composition may contain a polymerization inhibitor.

As the polymerization inhibitor, for example, a thermal polymerization inhibitor described in paragraph [0018] of JP4502784B can be used.

Specific examples of the polymerization inhibitor include p-methoxyphenol, phenothiazine, phenoxazine, and 4-methoxyphenol.

In a case where the resin composition contains a polymerization inhibitor, the resin composition may contain only one kind of the polymerization inhibitor, or may contain two or more kinds thereof.

In a case where the resin composition contains a polymerization inhibitor, a content of the polymerization inhibitor is not particularly limited, but for example, preferably 0.01% by mass to 3% by mass, more preferably 0.01% by mass to 1% by mass, and still more preferably 0.01% by mass to 0.8% by mass with respect to the total solid content mass of the resin composition.

The resin composition may further contain an acid generator, and the above-described resin may include an acid-decomposable resin. In a case where the resin composition contains an acid generator in addition to the compound represented by Formula (1) and the resin, and the resin includes an acid-decomposable resin, the resin composition can be used as a positive-tone resin composition.

### (Acid Generator)

The acid generator may be a photoacid generator or a thermal acid generator, but is preferably a photoacid generator. In a case where the compound represented by Formula (1) is heated to 230° C. or higher, the compound is colored, so that, in a case where the acid generator is a thermal acid generator, it is desirable that the thermal acid generator is a compound which is sensitive to heat lower than 230° C. and generates an acid.

The photoacid generator is a compound capable of generating an acid by irradiation with radiation such as ultraviolet rays, far ultraviolet rays, X-rays, and charged particle rays. The photoacid generator is preferably a compound which is sensitive to actinic rays having a wavelength of 300 nm or more, preferably 300 to 450 nm, and generates an acid. A photo-acid generator which is not directly sensitive to actinic rays having a wavelength of 300 nm or more can also be preferably used in combination with a sensitizer as long as it is a compound which is sensitive to actinic rays having a wavelength of 300 nm or more and generates an acid by being used in combination with the sensitizer.

The photo-acid generator is preferably a photo-acid generator which generates an acid with a pKa of 4 or less, more preferably a photo-acid generator which generates an acid with a pKa of 3 or less, and even more preferably a photo-acid generator which generates an acid with a pKa of 2 or less. The lower limit of pKa is not particularly limited, but is, for example, preferably −10 or more.

Examples of the photoacid generator include an ionic photoacid generator and a nonionic photoacid generator.

Examples of the ionic photoacid generator include an onium salt compound and a quaternary ammonium salt compound. Examples of the onium salt compound include a diaryliodonium salt compound and a triarylsulfonium salt compound.

The ionic photoacid generator is preferably an onium salt compound, and more preferably at least one selected from the group consisting of a diaryliodonium salt compound and a triarylsulfonium salt compound.

As the ionic photoacid generator, for example, ionic photoacid generators described in paragraphs [0114] to [0133] of JP2014-85643A can also be preferably used.

Examples of the nonionic photoacid generator include a trichloromethyl-s-triazine compound, a diazomethane compound, an imide sulfonate compound, and an oxime sulfonate compound.

Specific examples of the trichloromethyl-s-triazine compound, the diazomethane compound, and the imide sulfonate compound include compounds described in paragraphs [0083] to [0088] of JP2011-221494A.

Specific examples of the oxime sulfonate compound include compounds described in paragraphs [0084] to [0088] of WO2018/179640A.

The nonionic photoacid generator is, for example, preferably an oxime sulfonate compound from the viewpoint of sensitivity, resolution, and adhesiveness.

From the viewpoint of sensitivity and resolution, the photoacid generator is, for example, preferably at least one compound selected from the group consisting of an onium salt compound and an oxime sulfonate compound, and more preferably an oxime sulfonate compound.

In a case where the resin composition contains an acid generator, the resin composition may contain only one kind of the acid generator, or may contain two or more kinds thereof.

In a case where the resin composition contains an acid generator, a content of the acid generator is, for example, preferably 0.1% by mass to 10% by mass, and more preferably 0.5% by mass to 5% by mass with respect to the total solid content mass of the resin composition, from the viewpoint of sensitivity and resolution.

### (Acid-Decomposable Resin)

The acid-decomposable resin is a resin having an acid group protected by an acid-decomposable group.

The acid group protected by the acid-decomposable group in the acid-decomposable resin undergoes a deprotection reaction by a catalytic action of the acid generated by the acid generator to be an acid group. Due to the acid group, the film formed of the composition according to the present disclosure can be dissolved in, for example, a developer.

The acid-decomposable resin is preferably an addition polymerization-type resin and more preferably a polymer containing a constitutional unit derived from (meth)acrylic acid or an ester thereof. A constitutional unit other than the constitutional unit derived from (meth)acrylic acid or an ester thereof may include, for example, a constitutional unit derived from a styrene compound, a constitutional unit derived from a vinyl compound, and the like.

The acid group and the acid-decomposable group are not particularly limited.

Examples of the acid group include a carboxy group and a phenolic hydroxyl group.

Examples of the acid-decomposable group include a group which is relatively easily decomposed by the acid (for example, an acetal-type protective group such as a 1-alkoxyalkyl group, a tetrahydropyranyl group, and a tetrahydrofuranyl group) and a group which is relatively difficult to decompose by the acid (for example, a tertiary alkyl group such as a tert-butyl group, and a tertiary alkyloxycarbonyl group (so-called carbonic acid ester-type protective group) such as a tert-butyloxycarbonyl group).

The acid-decomposable group is preferably a group having a structure protected in a form of acetal (so-called acetal-type protective group).

From the viewpoint of suppressing variation in line width of a conductive wiring in a case of being applied to formation of a conductive pattern, the acid-decomposable group is preferably an acid-decomposable group having a formula weight of 300 or less.

From the viewpoint of resolution, an acid value of the acid-decomposable resin is preferably 0 mgKOH/g to 50 mgKOH/g, more preferably 0 mgKOH/g to 20 mgKOH/g, and still more preferably 0 mgKOH/g to 10 mgKOH/g.

The acid value of the resin in the present disclosure represents a mass of potassium hydroxide required to neutralize acidic components per 1 g of the resin. Specifically, the measurement is performed as follows.

The measurement sample is dissolved in a mixed solvent of tetrahydrofuran/water=9/1 (volume ratio). The obtained solution is subjected to neutralization titration with a 0.1 mol/L sodium hydroxide aqueous solution using a potentiometric titrator in an environment of an atmosphere temperature of 25° C. An inflection point of a titration pH curve is set as a titration end point, and the acid value is calculated from the following equation.

As the potentiometric titrator, a potentiometric titrator (model number: AT-510) manufactured by KYOTO ELECTRONICS MANUFACTURING CO., LTD. can be suitably used. However, the potentiometric titrator is not limited thereto.

$$A = 56.11 \times Vs \times 0.1 \times f / w$$

$$A : acid value ( mg KOH / g )$$

$$Vs : amount ( mL ) of the mol / L sodium hydroxide aqueous solution used for titration$$

$$f : titer of the 0.1 mol / L sodium hydroxide aqueous solution$$

$$w : mass ( g ) of the measurement \text{} sample ( expressed in terms of solid contents )$$

A weight-average molecular weight of the acid-decomposable resin is not particularly limited, but for example, preferably 2,000 to 60,000 and more preferably 3,000 to 50,000.

In a case where the resin composition contains an acid-decomposable resin, the resin composition may contain only one kind of the acid-decomposable resin, or may contain two or more kinds thereof.

In a case where the resin composition contains an acid-decomposable resin, a content of the acid-decomposable resin is, for example, preferably 50% by mass to 99.9% by mass, and more preferably 70% by mass to 98% by mass with respect to the total solid content mass of the resin composition.

### (Organic Solvent)

The resin composition may contain an organic solvent.

The organic solvent is not particularly limited, and examples thereof include an ester compound, an ether compound, a ketone compound, and an aromatic hydrocarbon compound.

With regard to details of these compounds, reference can be made to the description in WO2015/166779A, the contents of which are incorporated herein by reference.

Specific examples of the organic solvent include dichloromethane, chloroform, methyl 3-methoxypropionate, methyl 3-ethoxypropionate, ethyl 3-ethoxypropionate, ethyl acetate, butyl acetate, cyclohexyl acetate, ethyl cellosolve acetate, ethyl carbitol acetate, butyl carbitol acetate, ethyl lactate, 1-methoxy-2-propyl acetate, diethylene glycol dimethyl ether, 2-heptanone, cyclohexanone, cyclopentanone, propylene glycol monomethyl ether, 3-methoxy-N,N-dimethylpropanamide, and 3-butoxy-N,N-dimethylpropanamide.

In a case where the resin composition contains an organic solvent, the resin composition may contain only one kind of the organic solvent, or may contain two or more kinds thereof.

In a case where the resin composition contains an organic solvent, a content of the organic solvent is not particularly limited and can be appropriately set depending on the purpose.

A content of the organic solvent in the resin composition is preferably 10% by mass to 90% by mass, and more preferably 20% by mass to 80% by mass with respect to the total mass of the resin composition.

In a case where the resin composition is, for example, a film or a molded body, the content of the organic solvent in the resin composition is preferably 80% by mass or less, more preferably 70% by mass or less, and still more preferably 50% by mass or less with respect to the total mass of the resin composition.

### (Surfactant)

The resin composition may contain a surfactant.

In a case where the resin composition contains a surfactant, for example, the coating surface state in a case of coating and adhesiveness to a base material in a case of forming a film may be further improved.

Examples of the surfactant include surfactants described in paragraph [0017] of JP4502784B and paragraphs [0060] to [0071] of JP2009-237362A.

Examples of the surfactant include a fluorine-based surfactant, a silicone-based surfactant, a nonionic surfactant, an anionic surfactant, a cationic surfactant, and an amphoteric surfactant.

Among these, as the surfactant, at least one selected from the group consisting of a fluorine-based surfactant, a silicone-based surfactant, a nonionic surfactant, and an anionic surfactant is preferable.

As the fluorine-based surfactant, for example, it is preferable to use an acrylic compound, which has a molecular structure having a functional group including a fluorine atom and in which, by applying heat to the molecular structure, the functional group including a fluorine atom is broken to volatilize a fluorine atom. In addition, as the fluorine-based surfactant, for example, it is also preferable to use a copolymer of a fluorine atom-containing vinyl ether compound having a fluorinated alkyl group or a fluorinated alkylene ether group, and a hydrophilic vinyl ether compound. In addition, as the fluorine-based surfactant, for example, a block polymer can also be used. In addition, as the fluorine-based surfactant, for example, it is also preferable to use a fluorine-containing polymer compound including a repeating unit derived from a (meth)acrylate compound having a fluorine atom and a repeating unit derived from a (meth)acrylate compound having 2 or more (preferably 5 or more) alkyleneoxy groups [preferably an ethyleneoxy group and/or a propyleneoxy group]. In addition, as the fluorine-based surfactant, for example, a fluorine-containing polymer having an ethylenically unsaturated bond-containing group in the side chain can also be used.

A commercially available product can be used as the fluorine-based surfactant.

Examples of the commercially available product of the fluorine-based surfactant include: MEGAFACE (registered trademark) F-171, F-172, F-173, F-176, F-177, F-141, F-142, F-143, F-144, F-437, F-475, F-477, F-479, F-482, F-551-A, F-552, F-554, F-555-A, F-556, F-557, F-558, F-559, F-560, F-561, F-565, F-563, F-568, F-575, F-780, F-781-F, EXP.MFS-330, R-41, R-41-LM, R-01, R-40, R-40-LM, RS-43, TF-1956, RS-90, R-94, RS-718K, RS-72-K, RS-101, RS-102, and DS-21 [all of which are manufactured by DIC Corporation]; FLUORAD FC430, FC431, and FC171 [all of which are manufactured by Sumitomo 3M Ltd.]; SURFLON (registered trademark) S-382, SC-101, SC-103, SC-104, SC-105, SC-1068, SC-381, SC-383, S-393, and KH-40 [all of which are manufactured by Asahi Glass Co., Ltd.]; POLYFOX PF636, PF656, PF6320, PF6520, and PF7002 [all of which are manufactured by OMNOVA Solutions Inc.]; and FTERGENT (registered trademark) 710FM, 610FM, 601AD, 601ADH2, 602A, 215M, 245F, 251, 212M, 250, 209F, 222F, 208G, 710LA, 710FS, 730LM, 650AC, and 681 [all of which are manufactured by NEOS COMPANY LIMITED].

Examples of the silicone-based surfactant include a linear polymer including a siloxane bond and a modified siloxane polymer with an organic group introduced in the side chain and/or the terminal.

A commercially available product can be used as the silicone-based surfactant.

Examples of the commercially available product of the silicone-based surfactant include DC3PA, SH7PA, DC11PA, SH21PA, SH28PA, SH29PA, SH30PA, SH8400, and DOWSIL (registered trademark) 8032 ADDITIVE [all manufactured by Dow Corning Corporation]; X-22-4952, X-22-4272, X-22-6266, KF-351A, K354L, KF-355A, KF-945, KF-640, KF-642, KF-643, X-22-6191, X-22-4515, KF-6004, KP-341, KF-6001, and KF-6002 [all manufactured by Shin-Etsu Chemical Co., Ltd.]; F-4440, TSF-4300, TSF-4445, TSF-4460, and TSF-4452 [all manufactured by Momentive Performance Materials Japan LLC]; and BYK307, BYK323, and BYK330 [all manufactured by BYK-Chemie GmbH].

Examples of the nonionic surfactant include glycerol, trimethylolpropane, trimethylolethane, and ethoxylates thereof (for example, glycerol ethoxylate) and propoxylates thereof (for example, glycerol propoxylate).

In addition, examples of the nonionic surfactant include polyoxyethylene alkyl ether, polyoxyethylene alkyl aryl ether, a polyoxyethylene-oxypropylene block copolymer, sorbitan fatty acid ester, polyoxyethylene sorbitan fatty acid ester, polyoxyethylene sorbitol fatty acid ester, glycerin fatty acid ester, polyoxyethylene fatty acid ester, and polyoxyethylene alkylamine.

A commercially available product can be used as the nonionic surfactant.

Examples of the commercially available product of the nonionic surfactant include PLURONIC (registered trademark) L10, L31, L61, L62, 10R5, 17R2, and 25R2 [all manufactured by BASF SE]; TETRONIC (registered trademark) 304, 701, 704, 901, 904, and 150R1 [all manufactured by BASF SE]; Solsperse (registered trademark) 20000 [manufactured by Nippon Lubrizol Corporation]; NCW-101, NCW-1001, and NCW-1002 [all manufactured by FUJIFILM Wako Pure Chemical Corporation]; Pionin D-6112, D-6112-W, and D-6315 [all manufactured by TAKEMOTO OIL & FAT Co., Ltd.]; OLFINE (registered trademark) E1010 [all manufactured by Nissin Chemical Co., Ltd.], and SURFYNOL (registered trademark) 104, 400, and 440 [all manufactured by Nissin Chemical Co., Ltd.].

Examples of the anionic surfactant include a fatty acid salt, an alkyl sulfuric acid ester salt, an alkylbenzene sulfonate, an alkylnaphthalene sulfonate, a dialkyl sulfosuccinate, an alkyl diaryl ether disulfonate, an alkyl phosphate, a polyoxyethylene alkyl ether sulfate, a polyoxyethylene alkyl aryl ether sulfate, a naphthalenesulfonic formalin condensate, a polyoxyethylene alkyl phosphate ester salt, a glycerol borate fatty acid ester, and a polyoxyethylene glycerol fatty acid ester.

In a case where the resin composition contains a surfactant, the resin composition may contain only one kind of the surfactant, or may contain two or more kinds thereof.

In a case where the resin composition contains a surfactant, a content of the surfactant is not particularly limited, but for example, preferably 0.05% by mass to 3% by mass, more preferably 0.1% by mass to 2% by mass, and still more preferably 0.3% by mass to 1% by mass with respect to the total solid content mass of the resin composition.

### Other Components

The resin composition may contain a component other than the above-described components (so-called other components) as necessary, within a range in which the effect of the present disclosure is not impaired.

Examples of the other components include various additives.

Examples of the additive include a sensitizer, a colorant, a preservative, an antibacterial agent, and an antistatic agent.

### Step B

The step B is a step of heating the resin composition obtained in the step A.

In the step B, the resin composition is heated, whereby the compound represented by Formula (1) is colored to obtain a coloring composition. The obtained coloring composition has visible light shielding properties.

The method of heating the resin composition is not particularly limited, and a known heating method can be adopted.

The heating of the resin composition can be carried out using an oven, a hot plate, a heat roll, or the like.

From the viewpoint of more favorably coloring the resin composition black, a heating temperature of the resin composition is, for example, preferably 160° C. or higher and more preferably 180° C. or higher.

The upper limit of the heating temperature is not particularly limited, and may be, for example, a temperature at which the coloration is completed (for example, 260° C.).

A heating time of the resin composition is not particularly limited, and can be appropriately set, for example, according to the degree of coloration of the resin composition.

In a case where the resin composition has a form of a film or a molded body and the resin includes a thermoplastic resin, it is preferable to appropriately adjust the heating time, such as shortening the heating time, in consideration of whether or not the shape of the film or the molded body obtained in the step A can be maintained.

### [Method for Manufacturing Laminate for Display Element]

A method for manufacturing a laminate for a display element according to the present disclosure includes a step of producing a coloring composition by the above-described method for producing a coloring composition according to the present disclosure.

Since the method for producing a coloring composition according to the present disclosure is as described above, the description thereof will be omitted.

According to the method for manufacturing a laminate for a display element according to the present disclosure, for example, a laminate for a display element, including a black matrix (so-called black partition wall) having a favorable shape, can also be obtained.

The method for manufacturing a laminate for a display element according to the present disclosure may include a step (so-called other step) other than the step of producing a coloring composition by the method for producing a coloring composition according to the present disclosure.

### EXAMPLES

Hereinafter, the present disclosure will be described in detail according to Examples. However, the present disclosure is not limited to Examples below.

### [Synthesis of Compound]

The following compounds (1), (3), (5), (7), (8), and (65) were synthesized based on the following scheme. N—R in the scheme corresponds to Y² in Formula (1).

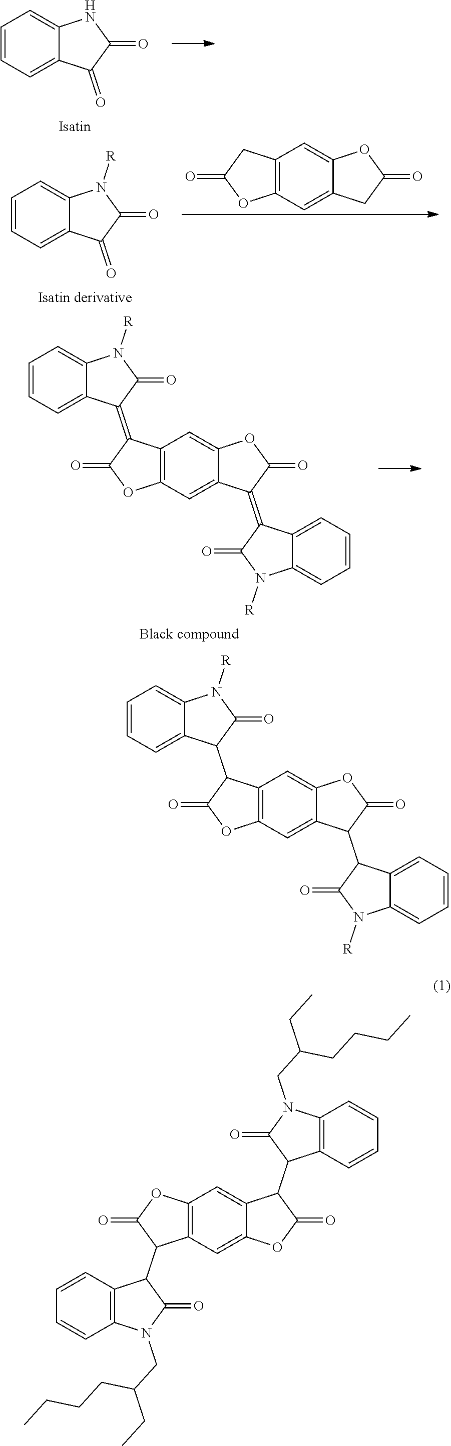

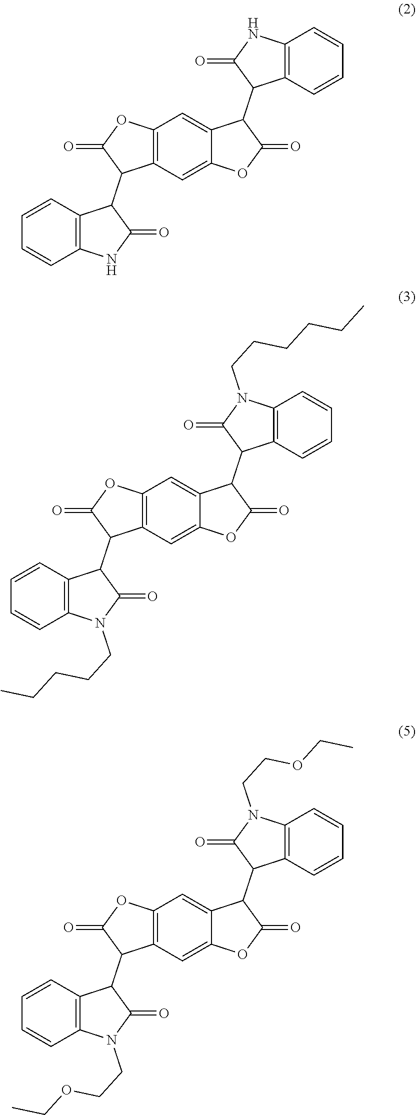

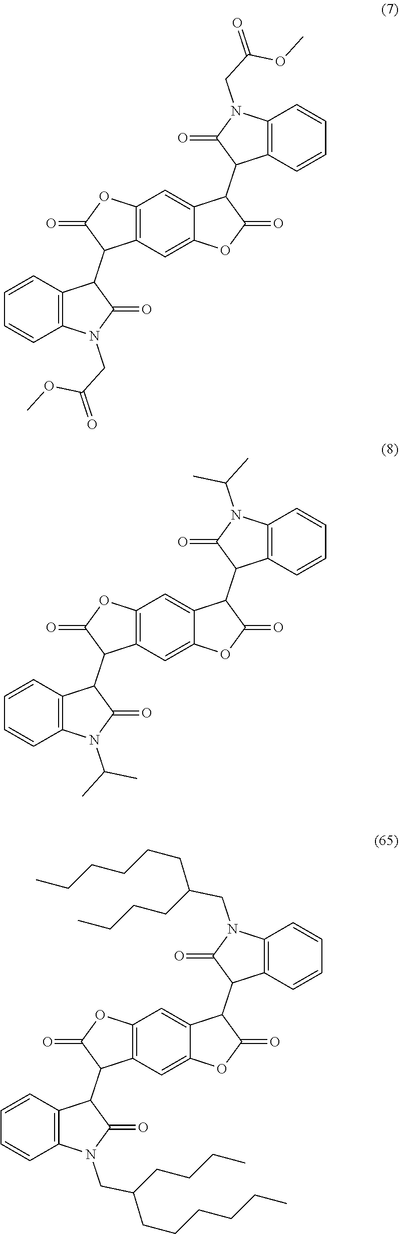 [SMILES: COC(=O)CN1C(=O)C(C2C(=O)Oc3cc4c(cc32)OC(=O)C4C2C(=O)N(CC(=O)OC)c3ccccc32)c2ccccc21 ; CC(C)N1C(=O)C(C2C(=O)Oc3cc4c(cc32)OC(=O)C4C2C(=O)N(C(C)C)c3ccccc32)c2ccccc21 ; CCCCCCC(CCCC)CN1C(=O)C(C2C(=O)Oc3cc4c(cc32)OC(=O)C4C2C(=O)N(CC(CCCC)CCCCCC)c3ccccc32)c2ccccc21]

### Example 1A: Synthesis of Compound (1)

With reference to J. Am. Chem. Soc. 2015, 137, pp. 15947 to 15956, a compound [the following compound (100)] corresponding to the black compound in the above scheme was synthesized using isatin as a starting material. 150 mL of tetrahydrofuran (THF) [containing a stabilizer, Wako 1st grade, manufactured by FUJIFILM Wako Pure Chemical Corporation] was charged into a 300 mL three-neck flask, and then 10.0 g of the synthesized compound (100) and 4.9 g of zine powder [Wako special grade. manufactured by FUJIFILM Wako Pure Chemical Corporation] were added thereto. The above-described three-neck flask was immersed in ice water to maintain the internal temperature at 5° C. or lower, and 15 mL of trifluoroacetic acid [Wako special grade, manufactured by FUJIFILM Wako Pure Chemical Corporation] was added dropwise thereto. After completion of the dropwise addition, the external equipment was removed, and the mixture was reacted in a water bath for 2 hours such that the internal temperature did not reach 40° C. or higher. The reaction solution was filtered through Celite, 10 mL of ultrapure water was added to the filtrate, and the solution was heated to 40° C. to distill off THF under reduced pressure. The precipitated gray solid was subjected to suction filtration and washed with 300 mL of ultrapure water. The resultant was dried for 12 hours using a blast dryer at a set temperature of 50° C., thereby obtaining 4.5 g of a compound (1) (yield: 46%).

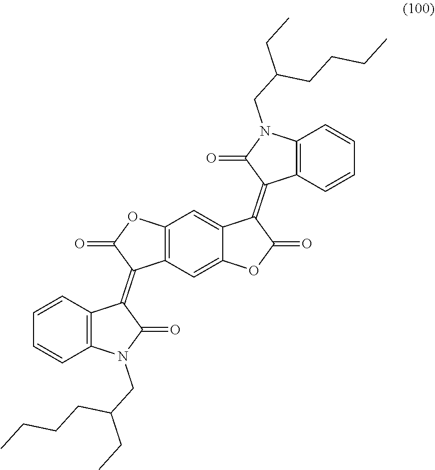 [SMILES: CCCCC(CC)CN1C(=O)/C(=C2/C(=O)Oc3cc4c(cc32)OC(=O)/C4=C2/C(=O)N(CC(CC)CCCC)c3ccccc32)c2ccccc21]

A structure of the obtained compound (1) was confirmed to be the structure of the above-described compound (1) by ¹H-NMR. NMR data of the obtained compound (1) are shown below.

### <NMR Data of Compound (1)>

¹H-NMR (CDCl₃-d) 6=0.80 to 1.90 (m, 30H), 3.30 to 3.70 (m, 4H), 4.20 to 4.50 (s×3, 4H), 6.20 to 6.50 (m, 2H), 6.80 to 7.20 (m, 6H), 7.30 to 7.40 (m, 2H)

### Example 2A: Synthesis of Compound (3)

With reference to Journal of Medicinal Chemistry, 2008, 51, pp. 4932 to 4947, an isatin derivative was synthesized by reacting isatin with 1-bromohexane. Using the synthesized isatin derivative, a compound (3) was synthesized by the same method as that for the compound (1) described above.

A structure of the obtained compound (3) was confirmed to be the structure of the above-described compound (3) by ¹H-NMR. NMR data of the obtained compound (3) are shown below.

### <NMR Data of Compound (3)>

¹H-NMR (CDCl₃-d) δ=0.69 to 0.71 (t, 6H), 3.35 to 3.76 (m, 12H), 4.20 to 4.50 (s×3, 4H), 6.20 to 6.50 (m, 2H), 6.80 to 7.20 (m, 6H), 7.30 to 7.40 (m, 2H)

### Example 3A: Synthesis of Compound (5)

A compound (5) was synthesized in the same manner as in the compound (3), except that in the synthesis of the compound (3), “2-bromoethyl ethyl ether” was used instead of “1-bromohexane”.

A structure of the obtained compound (5) was confirmed to be the structure of the above-described compound (5) by ¹H-NMR. NMR data of the obtained compound (5) are shown below.

### <NMR Data of Compound (5)>

¹H-NMR (CDCl₃-d) δ=0.86 to 0.90 (t, 6H), 3.65 to 3.90 (m, 12H), 4.20 to 4.50 (s×3, 4H), 6.20 to 6.50 (m, 2H), 6.80 to 7.20 (m, 6H), 7.30 to 7.40 (m, 2H)

### Example 4A: Synthesis of Compound (7)

With reference to Chemistry-A European Journal, 2021, 27, pp. 4302 to 4306, an isatin derivative was synthesized by reacting isatin with methyl bromoacetate. Using the synthesized isatin derivative, a compound (7) was synthesized by the same method as that for the compound (1) described above.

A structure of the obtained compound (7) was confirmed to be the structure of the above-described compound (7) by ¹H-NMR. NMR data of the obtained compound (7) are shown below.

### <NMR Data of Compound (7)>

¹H-NMR (CDCl₃-d) δ=3.49 to 3.53 (t, 6H), 4.20 to 4.50 (m, 8H), 6.20 to 6.50 (m, 2H), 6.80 to 7.20 (m, 6H), 7.30 to 7.40 (m, 2H)

### Example 5A: Synthesis of Compound (8)

With reference to Org. Lett., 2021, 23, pp. 2273 to 2278, an isatin derivative was synthesized by reacting isatin with 2-iodopropane. Using the synthesized isatin derivative, a compound (8) was synthesized by the same method as that for the compound (1) described above.

A structure of the obtained compound (8) was confirmed to be the structure of the above-described compound (8) by ¹H-NMR. NMR data of the obtained compound (8) are shown below.

### <NMR Data of Compound (8)>

¹H-NMR (CDCl₃-d) δ=1.17 to 1.21 (m, 12H), 3.60 to 3.70 (m, 2H), 4.20 to 4.50 (s×3, 4H), 6.20 to 6.50 (m, 2H), 6.80 to 7.20 (m, 6H), 7.30 to 7.40 (m, 2H)

### Example 6A: Synthesis of Compound (2)

In the above scheme, a black compound [a compound (101) having the following structure] was directly synthesized from isatin without going through an isatin derivative. 100 mL of N,N-dimethylformamide (DMF) [Wako special grade, manufactured by FUJIFILM Wako Pure Chemical Corporation] was charged into a 300 mL three-neck flask, and then 10.0 g of a compound (101) and 1.0 g of Palladium 10% on Carbon [product name, manufactured by Tokyo Chemical Industry Co., Ltd.] were further added thereto. A balloon in which nitrogen was sealed was attached to the flask, the inside of the flask was degassed, and then the inside of the flask was purged with nitrogen. The mixture was reacted at room temperature for 2 hours in a nitrogen atmosphere. The reaction solution was filtered through Celite, and the Celite was washed with ethyl acetate. The filtrate was subjected to an evaporator in a water bath at 40° C. to distill off ethyl acetate, thereby obtaining a DMF solution containing a target substance. The DMF solution was purified by silica gel column chromatography, and a fraction containing the target substance was subjected to the evaporator again to obtain 10 mg (yield: 0.22%) of a precipitated gray compound (2).

A structure of the obtained compound (2) was confirmed to be the structure of the above-described compound (2) by ¹H-NMR. NMR data of the obtained compound (2) are shown below.

### <NMR Data of Compound (2)>

¹H-NMR (DMSO-d₆) δ=4.25 to 4.40 (m, 2H), 4.75 to 4.84 (m, 2H), 6.47 to 6.55 (m, 1H), 6.70 to 7.00 (m, 7H), 7.10 to 7.32 (m, 2H)

### Example 7A: Synthesis of Compound (65)

A compound (65) was obtained in the same manner as in the compound (1), except that in the synthesis of the compound (1), “5-(bromomethyl)undecane” was used instead of “2-ethylhexyl bromide”.

A structure of the obtained compound (65) was confirmed to be the structure of the above-described compound (65) by ¹H-NMR. NMR data of the obtained compound (65) are shown below.

### <NMR Data of Compound (65)>

¹H-NMR (CDCl₃) δ=0.83 to 0.89 (m, 12H), 0.90 to 1.50 (m, 32H), 1.60 to 1.78 (brs×2, 2H), 3.38 to 3.60 (m, 4H), 4.25 to 4.48 (m, 4H), 6.23 to 6.50 (m, 2H), 6.82 to 7.07 (m, 6H), 7.33 to 7.40 (m, 2H)

### [Measurement of Absorption Spectrum]

1.1 mg of the compound (100) and 1.1 mg of the compound (1) were respectively dissolved in 50 mL of tetrahydrofuran (THF) [containing a stabilizer, Wako 1st grade, manufactured by FUJIFILM Wako Pure Chemical Corporation] to prepare a THF solution of the compound (100) and a THF solution of the compound (1).

1.1 mg of the compound (1) was heated for 60 minutes in an environment of an atmosphere temperature of 230° C. The obtained compound is referred to as “compound (1H)”. Next, the compound (1H) was dissolved in 50 mL of tetrahydrofuran (THF) [containing a stabilizer, Wako 1st grade, manufactured by FUJIFILM Wako Pure Chemical Corporation] to prepare a THF solution of the compound (1H).

The THF solution of the compound (100), the THF solution of the compound (1), and the THF solution of the compound (1H) prepared as described above were each placed in a 1 cm cell, and using a spectrophotometer [product name: U-4100, manufactured by Hitachi, Ltd.] as a measuring device, an absorption spectrum was measured to obtain a molar absorption coefficient (E) and a maximal absorption wavelength (λmax).

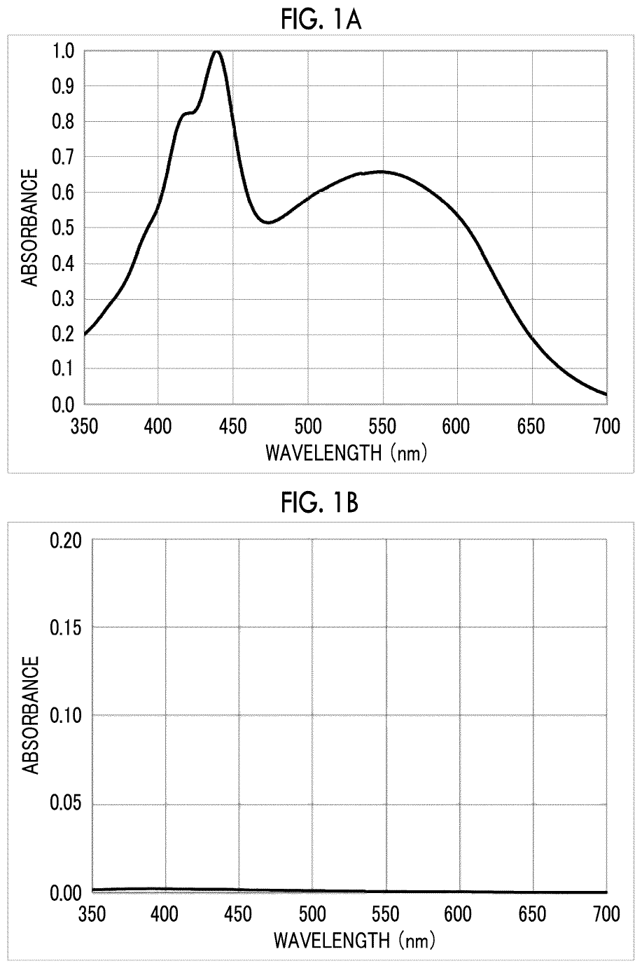

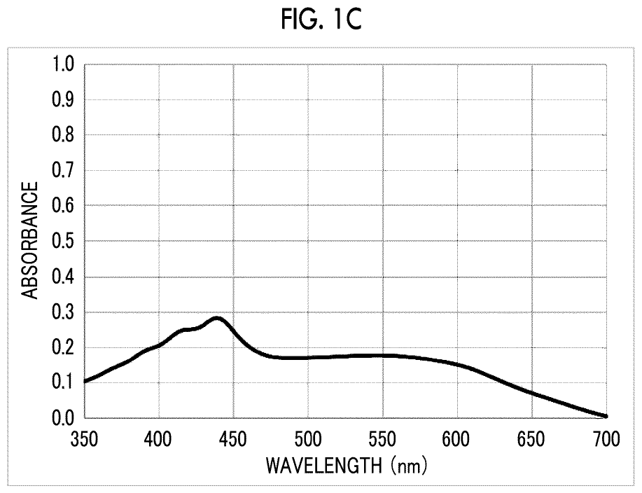

The absorption spectrum of the THF solution of the compound (100), the absorption spectrum of the THF solution of the compound (1), and the absorption spectrum of the THF solution of the compound (1H) are shown in FIGS. 1A, 1B, and 1C, respectively.

The molar absorption coefficient of the THF solution of the compound (1) was less than 2,000 L/(mol·cm) in a wavelength range of 400 nm to 780 nm. The molar absorption coefficient of the THF solution of the compound (1H) was 2,000 L/(mol·cm) or more in the entire wavelength range of 400 nm to 650 nm.

### Example 1B

### 1. Preparation of Composition

A composition having the following formulation was prepared. Specifically, the preparation was carried out as follows.

The following components other than the compound (1) were mixed to obtain a mixture. 28 g of the compound (1) [an amount of 20 parts by mass in a case where the total solid content mass of the composition to be finally obtained is set to 100 parts by mass] was added to the obtained mixture, and then 28 g of zirconia beads having a diameter of 0.5 mm were added thereto and shaken for 2 hours to obtain a composition.

-Formulation of composition-

| | |
|---|---|
| Compound (1) | 28 g |
| Resin | 70 g |
| [benzyl methacrylate/methacrylic acid | |
| copolymer (monomer molar ratio: 70/30), | |
| alkali-soluble resin, binder] | |
| Polymerizable monomer | 35 g |
| [dipentaerythritol hexaacrylate] | |
| Polymerization inhibitor | 0.26 g |
| [p-Methoxyphenol] | |
| Surfactant | 0.83 g |
| [product name: MEGAFACE (registered | |
| trademark) F-781-F, fluorine-based surfactant, | |
| manufactured by DIC Corporation] | |
| Photopolymerization initiator | 5.8 g |
| [IRGACURE (registered trademark) OXE01, | |
| manufactured by BASF SE] | |
| Organic solvent (1) | 202 g |
| [propylene glycol monomethyl ether acetate] | |
| Organic solvent (2) | 52 g |
| [methyl ethyl ketone] | |

### 2. Formation of Cured Film

The composition was applied onto a soda glass substrate using a spin coater to form a coating film having a film thickness of 22 km. Next, the coating film was exposed through a photo mask using a high-pressure mercury lamp (main wavelength: 365 nm) at an exposure amount of 200 mJ/cm². Next, the exposed coating film was subjected to shower development using a 0.04% by mass potassium hydroxide aqueous solution at 23° C. Next, the coating film after the shower development was post-baked at 230° C. for 1 hour to form a cured film having a film thickness of 20 m on the soda glass substrate.

### 3. Evaluation

### (1) Light Shielding Properties

Light shielding properties of the cured film were evaluated. The light shielding properties of the cured film were determined using an optical density (OD) of the cured film as an indicator.

Using an ultraviolet-visible-near infrared spectrophotometer [model number: UV-3600i Plus, manufactured by Shimadzu Corporation] as a measuring device, the optical density of the cured film was measured. Based on the obtained measured value of the optical density, the light shielding properties of the cured film were evaluated according to the following evaluation standard. In a case where the evaluation result was “AA” or “A”, it was determined that there was no problem in practical use. The results are shown in Table 1.

As the optical density of the cured film was higher, the light shielding properties of the cured film was better, and in other words, it means that a visible light shielding effect of a coloring agent contained in the cured film [for example, in the case of Example 1B, a coloring agent formed by heating the compound (1) which was a coloring agent precursor] was high.

### (Evaluation Standard)

AA: optical density was 3.0 or more.
A: optical density was 1.5 or more and less than 3.0.
B: optical density was less than 1.5.

### (2) Pattern Shape

A cross section of the cured film was observed using a scanning electron microscope (SEM), “width of the uppermost portion” and “width of the lowermost portion” of the cured film were measured, and “(Width of uppermost portion)−(Width of lowermost portion)” was determined. Based on the obtained value, the pattern shape of the cured film was evaluated according to the following evaluation standard.

(Evaluation standard)
A: (Width of uppermost portion)−(Width of lowermost portion)<2.0 μm
B: (Width of uppermost portion)−(Width of lowermost portion)≥2.0 μm

In a case where UV transmittance of the coating film of the composition was low, film thickening occurred, and the width of the upper portion of the cured film increased. In addition, in a case where the UV transmittance of the coating film of the composition was low, UV light did not sufficiently reach the deep portion of the coating film, curing was insufficient, and an undercut occurred during the development, so that the width of the lower portion of the cured film decreased. As a result, the difference between the width of the upper portion and the width of the lower portion of the cured film was large, and the pattern did not have a favorable shape. On the other hand, in a case where the UV transmittance of the coating film of the composition was high, the above-described phenomenon was less likely to occur, and thus a pattern having a favorable shape could be obtained. The fact that the pattern having a favorable shape could be formed means that the coating film had excellent UV transmittance, and in other words, it means that the coloring agent precursor [for example, the compound (1) in Example 1] did not or hardly did not hinder the UV transmittance of the coating film.

### Examples 2B to 5B and Example 7B

### 1. Preparation of Composition

In Examples 2B, 3B, 4B, 5B, and 7B, the same operation as in Example 1B was performed to obtain compositions, except that the “compound (1)” in Example 1B was changed to each of the “compound (3)”, “compound (5)”, “compound (7)”, “compound (8)”, and “compound (2)”.

### 2. Formation of Cured Film

The same operation as in Example 1B was performed to form a cured film.

### 3. Evaluation

The evaluations of “(1) Light shielding properties” and “(2) Pattern shape” were performed in the same manner as in Example 1B.

### Example 6B

### 1. Preparation of Composition

In Example 6B, a composition was obtained by performing the same operation as in Example 1B, except that the blending amount of the compound (1) was changed from “20 parts by mass” to “10 parts by mass”.

### 2. Formation of Cured Film

In Example 6B, the same operation as in Example 1B was performed to form a cured film, except that the composition was applied onto a soda glass substrate using a spin coater to form a coating film having a film thickness of 32 μm, and the film thickness of the cured film was set to “30 μm”.

### 3. Evaluation

The evaluations of “(1) Light shielding properties” and “(2) Pattern shape” were performed in the same manner as in Example 1B.

### Comparative Example 1B

### 1. Preparation of Composition

In Comparative Example 1B, the same operation as in Example 1B was performed to obtain a composition, except that the “compound (1)” in Example 1B was changed to “compound (101) [trade name: Irgaphor (registered trademark) Black S 0100 CF, black pigment, manufactured by BASF SE]” having the following structure.

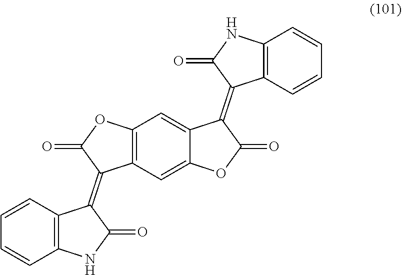 [SMILES: O=C1Nc2ccccc2/C1=C1\C(=O)Oc2cc3c(cc21)OC(=O)/C3=C1/C(=O)Nc2ccccc21]

### 2. Formation of Cured Film

The same operation as in Example 1B was performed to form a cured film.

### 3. Evaluation

The evaluations of “(1) Light shielding properties” and “(2) Pattern shape” were performed in the same manner as in Example 1B.

### Comparative Example 2B

### 1. Preparation of Composition

As a composition of Comparative Example 2B, a composition of Example 11 disclosed in JP2019-179111A was used. A method for preparing the composition was the same as in Example 11 disclosed in the above publication.

### 2. Formation of Cured Film

A cured film was formed by performing the same operation as the operation disclosed in paragraph [0135] of JP2019-179111A, except that the UV irradiation amount was changed from “50 J/m²” to “200 mJ/cm²” and the film thickness was changed from “10 m” to “20 m”.

### 3. Evaluation

The evaluations of “(1) Light shielding properties” and “(2) Pattern shape” were performed in the same manner as in Example 1B.

### Comparative Example 3B

### 1. Preparation of Composition

In Comparative Example 3B, the same operation as in Example 6B was performed to obtain a composition, except that the “compound (1)” in Example 6B was changed to the “compound (101) [trade name: Irgaphor (registered trademark) Black S 0100 CF, black pigment, manufactured by BASF SE]” having the above-described structure.

### 2. Formation of Cured Film

The same operation as in Example 1B was performed to form a cured film.

### 3. Evaluation

The evaluations of “(1) Light shielding properties” and “(2) Pattern shape” were performed in the same manner as in Example 1B.

TABLE 1

| | Compound | | | Evaluation | |
|---|---|---|---|---|---|
| | | Blending amount | Film thickness | Light shielding | |
| | Type | [part by mass] | [μm] | properties | Pattern shape |
| Example 1B | Compound (1) | 20 | 20 | AA | A |
| Example 2B | Compound (3) | 20 | 20 | AA | A |
| Example 3B | Compound (5) | 20 | 20 | AA | A |
| Example 4B | Compound (7) | 20 | 20 | AA | A |
| Example 5B | Compound (8) | 20 | 20 | AA | A |
| Example 6B | Compound (1) | 10 | 30 | A | A |
| Example 7B | Compound (2) | 10 | 30 | A | A |
| Comparative | Compound (101) | 20 | 20 | AA | B |
| Example 1B | | | | | |
| Comparative | Carbon black | 4.6 | 20 | AA | B |
| Example 2B | | | | | |
| Comparative | Compound (101) | 10 | 30 | A | B |
| Example 3B | | | | | |

In Table 1, the blending amount of the compound indicates an amount in a case where the total solid content mass of the composition is set to 100 parts by mass.

As shown in Table 1, in a case where the compound (1), the compound (2), the compound (3), the compound (5), the compound (7), and the compound (8), which are the compound according to the present disclosure, were used, a pattern having a favorable shape could be formed. On the other hand, in a case where the compound (101) which is not the compound according to the present disclosure and carbon black were used, a pattern having a favorable shape could not be formed.

In addition, as shown in Table 1, it was found that, with the compound according to the present disclosure, a pattern having a favorable shape could be formed even in a thick film of 30 ram.

From the results shown in Table 1, it was found that the compound according to the present disclosure could achieve both transmission and shielding of light.

The disclosure of Japanese Patent Application No. 2022-106689 filed on Jun. 30, 2022 and the disclosure of Japanese Patent Application No. 2023-015690 filed on Feb. 3, 2023 are incorporated in the present specification by reference.

All documents, patent applications, and technical standards described in the present specification are incorporated herein by reference to the same extent as in a case of being specifically and individually noted that individual documents, patent applications, and technical standards are incorporated by reference.

What is claimed is:

1. A compound represented by Formula (1), 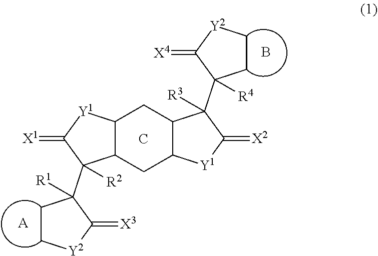
in Formula (1), X¹, X², X³, X⁴, Y¹, and Y² each independently represent an oxygen atom, a sulfur atom, or N-L¹, L¹ represents a hydrogen atom, an alkyl group, an acyl group, an alkoxycarbonyl group, or an aminocarbonyl group, R¹, R², R³, and R⁴ each independently represent a hydrogen atom, —O-L², —OCO-L³, —S-L², or —OSO-L³, L² represents a hydrogen atom or an alkyl group and L³ represents an alkyl group or an amino group, provided that at least one of R¹ or R² represents a hydrogen atom and at least one of R³ or R⁴ represents a hydrogen atom, and A, B, and C each independently represent an aromatic ring.

2. The compound according to claim 1,
wherein, in Formula (1), X¹, X², X³, and X⁴ are oxygen atoms, one of R¹ or R² is a hydrogen atom and the other is a hydroxy group, and one of R³ or R⁴ is a hydrogen atom and the other is a hydroxy group.

3. The compound according to claim 1,
wherein, in Formula (1), X¹, X², X³, and X⁴ are oxygen atoms, and R¹, R², R³, and R⁴ are hydrogen atoms.

4. The compound according to claim 1,
wherein the compound is a coloring agent precursor.

5. A composition comprising:
a compound represented by Formula (1), 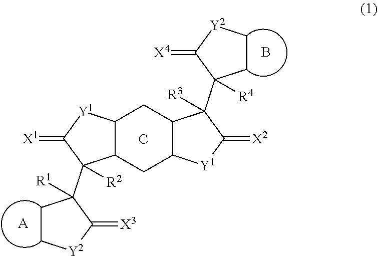
in Formula (1), X¹, X², X³, X⁴, Y¹, and Y² each independently represent an oxygen atom, a sulfur atom, or N-L¹, L¹ represents a hydrogen atom, an alkyl group, an acyl group, an alkoxycarbonyl group, or an aminocarbonyl group, R¹, R², R³, and R⁴ each independently represent a hydrogen atom, —O-L², —OCO-L³, —S-L², or —OSO-L³, L² represents a hydrogen atom or an alkyl group and L³ represents an alkyl group or an amino group, provided that at least one of R¹ or R² represents a hydrogen atom and at least one of R³ or R⁴ represents a hydrogen atom, and A, B, and C each independently represent an aromatic ring.

6. The composition according to claim 5, further comprising:
a resin.

7. A film comprising:
a compound represented by Formula (1), 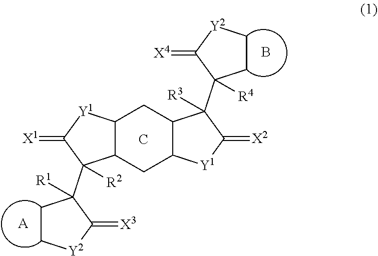
in Formula (1), X¹, X², X³, X⁴, Y¹, and Y² each independently represent an oxygen atom, a sulfur atom, or N-L¹, L¹ represents a hydrogen atom, an alkyl group, an acyl group, an alkoxycarbonyl group, or an aminocarbonyl group, R¹, R², R³, and R⁴ each independently represent a hydrogen atom, —O-L², —OCO-L³, —S-L², or —OSO-L³, L² represents a hydrogen atom or an alkyl group and L³ represents an alkyl group or an amino group, provided that at least one of R¹ or R² represents a hydrogen atom and at least one of R³ or R⁴ represents a hydrogen atom, and A, B, and C each independently represent an aromatic ring.

8. A method for producing a coloring composition, comprising:
a step A of obtaining a composition containing a compound represented by Formula (1) and a resin; and
a step B of heating the composition, 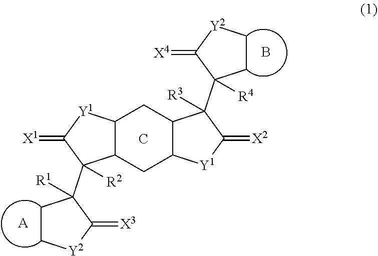
in Formula (1), X¹, X², X³, X⁴, Y¹, and Y² each independently represent an oxygen atom, a sulfur atom, or N-L¹, L¹ represents a hydrogen atom, an alkyl group, an acyl group, an alkoxycarbonyl group, or an aminocarbonyl group, R¹, R², R³, and R⁴ each independently represent a hydrogen atom, —O-L², —OCO-L³, —S-L², or —OSO-L³, L² represents a hydrogen atom or an alkyl group and L³ represents an alkyl group or an amino group, provided that at least one of R¹ or R² represents a hydrogen atom and at least one of R³ or R⁴ represents a hydrogen atom, and A, B, and C each independently represent an aromatic ring.

9. The method for producing a coloring composition according to claim 8,
wherein the composition further contains a polymerization initiator.

10. The method for producing a coloring composition according to claim 9,
wherein the composition further contains a polymerizable monomer.

11. The method for producing a coloring composition according to claim 8,
wherein the composition further contains an acid generator, and
the resin includes an acid-decomposable resin.

12. A method for manufacturing a laminate for a display element, comprising:
a step of producing a coloring composition by the method for producing a coloring composition according to claim 8.
